(19) United States
(12) Patent Application Publication
(10) Pub. No.: US 2026/0009001 A1
(43) Pub. Date: Jan. 8, 2026

(54) SYSTEMS AND METHODS FOR APPLYING MECHANICAL FORCE TO LIVING CELLS TO GENERATE A PHENOTYPIC RESPONSE

(71) Applicant: Kite Pharma, Inc., Santa Monica, CA (US)

(72) Inventors: Jason Lee, Corona, CA (US); Nathaniel W. Freund, Los Angeles, CA (US)

(21) Appl. No.: 19/260,894

(22) Filed: Jul. 7, 2025

Related U.S. Application Data
(60) Provisional application No. 63/668,671, filed on Jul. 8, 2024.

(51) Int. Cl. C12N 5/0783; C12M 1/00; C12M 1/42; C12N 15/86

(52) U.S. Cl. CPC C12N 5/0636; C12M 29/14; C12M 35/04; C12N 15/86; C12N 2510/00; C12N 2521/00; C12N 2740/10043; C12N 2740/15043

(57) ABSTRACT

The present disclosure describes technology for applying mechanical stress to cells to improve cell population phenotype for cell therapy products. More specifically, the disclosure describes optimized methods of applying shear stress to immune cells leading to improved activation, expansion, and transduction resulting in healthier engineered immune cell populations (e.g., increased naïve cells and central memory cells).

### CROSS-REFERENCE TO RELATED APPLICATIONS

This application claims priority to U.S. Provisional Application No. 63/668,671, filed on Jul. 8, 2024 the entirety of which is incorporated herein by reference.

### FIELD

The present disclosure relates to the field of cell therapy, and more specifically, to systems and methods to generate more robust T cell activation, transduction, and expansion leading to improved phenotypic outcomes of a cell therapy product.

### BACKGROUND

An ongoing challenge in the cell therapy industry involves discovery and implementation of systems and methods that can optimize a cell therapy manufacturing process. Current equipment and methods used in the cell therapy industry are often inadequate and/or suboptimal because they may often be adapted from other fields such as stem cell research and biologics. Furthermore, the relationship between equipment design and cell product outcome is relatively unknown and not well characterized making development of improved systems and methods difficult. This lack of understanding often leads to excessive technology development times throughout the design-build-test-reiterate cycles leading to high implementation costs and hesitation of adoption. Controlling the outcome of a desirable cell product quality as a function of equipment design, i.e. software parameters, hardware selection, method workflows, and single-use kit fluid path, is an area of underutilized development methodology which the technology described herein addresses.

Further, equipment development and optimization may often not a priority for pharmaceutical companies attempting to receive regulatory approval for a product. As of the beginning of 2024, only six FDA approved CAR-T products exist and are autologous. The industry has focused a lot of effort on throughput in a scale-out strategy of autologous cell therapy. Each lot produced is patient specific and the target dosage can be reached in less than a week with a simple cell therapy process. Many allogenic cell therapies are in the clinical trial phase, but none are commercially available at the time of this writing. Preliminary data shows that these human immune cells may be sensitive to their environment and that existing equipment such as upright stirred tank reactors may do little to account for in vivo environmental conditions that can be optimal for cell health especially in the autologous and allogeneic setting.

Immune cells in the human body may be subjected to a microenvironment that may be vastly different than in the laboratory setting. Similar to stem cells, human immune cells have complex interactions with their environment and small changes can influence their performance and ultimately the clinical outcome. Off-the-shelf commercially available equipment for the cell therapy landscape does not account for the specific requirements of human immune cells.

Historically, shear stress may be often credited as one of the major challenges of scaling-up traditional mammalian cell culture and increased shear stress may often be currently seen as negatively impacting cell health. As such, equipment manufacturers have worked to develop systems and methods that minimize mechanical force application (e.g., shear stress) on a cell culture. Further, not only do cell therapy equipment manufactures fail to consider, or even acknowledge, the importance of replicating the microenvironment in which human immune cells are extracted from, but they have actually worked to reduce and eliminate natural conditions for the cells (e.g., the existence of mechanical forces such as shear stress). This may be a major problem and often contributes the various manufacturing issues and variable outcomes leading to less robust T cell activation, transduction, and expansion and ultimately fewer naïve and central memory cells in a cell therapy product. As cell therapy processes become more automated to achieve scale-out capacity targets, the problem of equipment design decisions leading to unintended and unknown effects become more pronounced, and thus important to overcome through novel processing methods. Again, the technology described herein address these and other unmet needs in the field.

The technologies disclosed herein address these and other problems within the field. The technologies described herein may be applicable to both allogenic and autologous cell therapy products. Specifically, the systems and methods described herein may be used to apply mechanical forces (e.g., shear force) to cells in a way that may increase the robustness of T cell activation, transduction, and expansion among other things. Unlike existing technology, the technology described herein may optimize T cell and CAR-T cell manufacturing systems and methods to mimic the microenvironment found in the human body resulting in advantages such as a more robust T cell activation, transduction, and expansion. Counterintuitively, instead of reducing exposure to shear stresses, the systems and methods described herein may expose human immune cells to similar shear stresses found in the human body. The result may be generation of more naïve cell and central memory phenotypes which may lead to better clinical outcomes.

### SUMMARY

In various aspects, a method of applying a shear force to a first cell population to cause a subsequent phenotypic response is described according to various embodiments.

In various embodiments, the method comprises delivering a fluid comprising a first cell population to a mechanical force generating system.

In various embodiments, the method comprises applying a shear force to the first cell population using the mechanical force generating system to create a second cell population. In various embodiments, the step of applying the shear force causes a subsequent phenotypic response.

In various embodiments, the method comprises activating the second cell population to create an activated cell population.

In various embodiments, the method comprises transducing the activated cell population to create a final cell population.

In various embodiments, the method comprises harvesting the final cell population to generate a cell therapy product, wherein the subsequent phenotypic response comprises an increase of central memory or naïve cells in the final cell population. In various embodiments, the increase of central memory cells is 0.3% to 16.3% and the increase of naïve cells is 0.3% to 2.5%.

In various embodiments, the subsequent phenotypic response comprises a decrease of effector cells in the final cell population. In various embodiments, the decrease of effector cells is 0.2% to 12.9%.

In various embodiments, the subsequent phenotypic response comprises an increase of T cells and a decrease of both B cells and NK cells in the final cell population. In various embodiments, the decrease of B cells is 0.3% to 4.5%, and the decrease of NK cells is 0.3% to 2.0%.

In various embodiments, the subsequent phenotypic response comprises an increase of CD69 or CD25 expression in the activated cell population. In various embodiments, the increase of CD69 is 2% to 4% and the increase of CD25 is 5% to 14%.

In various embodiments, the subsequent phenotypic response comprises an increase of PD-1 expression. In various embodiments, the increase of PD-1 expression is 0.6% to 7.1%.

In various embodiments, the subsequent phenotypic response comprises an increase of CD27 or CD28 expression. In various embodiments, the increase of CD27 expression is 0.3% to 3.9% and the increase of CD28 expression is 0.3% to 3.4%.

In various embodiments, the step of transducing comprises using a lentiviral vector or a retroviral vector.

In various embodiments, the first cell population comprises T cells. In various embodiments, the first cell population comprises peripheral blood mononuclear cells (PBMCs).

In various embodiments, the step of applying the shear force occurs for 30 minutes to 120 minutes. In various embodiments, the step of applying the shear force occurs for 60 minutes to 120 minutes.

In various embodiments, the step of applying the shear force comprises applying at a shear rate of 569.78 s⁻¹ to 10,533.53 s⁻¹. In various embodiments, the step of applying the shear force comprising applying at a shear rate of 614.75 s⁻¹.

In various embodiments, the step of applying the shear force comprises applying a total shear exposure of 1,106,557 s to 4,426,230 s.

In various embodiments, the mechanical force generating system comprises a housing for storing the cell population. In various embodiments, the mechanical force generating system comprises a fluid channel having a first end and a second end, wherein the first and second ends are fluidically connected to the housing. In various embodiments, the mechanical force generating system comprises a pump positioned along the fluid channel or in the housing.

In various embodiments, the inner diameter of the fluid channel is 4.8 mm to 25.4 mm. In various embodiments, the length of the fluid channel is 45 cm.

In various embodiments, the pump operates to produce a fluid flow rate of 400 mL/min to 55,000 mL/min.

In various embodiments, the pump comprises a peristaltic pump.

In various aspects, an improved cell therapy product made by delivering a fluid comprising a first cell population to a mechanical force generating system, applying a shear force to the first cell population using the mechanical force generating system to create a second cell population, wherein the step of applying of the shear force causes a subsequent phenotypic response, activating the second cell population to create an activated cell population, transducing the activated cell population to create a final cell population, and harvesting a final cell population to generate a cell therapy product, wherein the subsequent phenotypic response comprises an increase of central memory or naïve cells in the final cell population is described.

In various aspects, an improved cell therapy product is described. In various embodiments, the improved cell therapy product comprises a population of shear force treated cells having an increase of CD69 expression, CD25 expression, PD-1 expression. CD27 expression, CD28 expression, IL-2 expression, CD4 expression, CD8 expression, naïve T-cells, central memory T cells, central memory T cells, or a combination thereof.

In various aspects, a method of applying a shear force to a first cell population to cause a subsequent phenotypic response is described. In various embodiments, the method comprises delivering a fluid comprising a first cell population to a mechanical force generating system. In various embodiments, the method comprises step for applying a shear force to the first cell population using the mechanical force generating system to create a second cell population. In various embodiments, the step for applying the shear force causes a subsequent phenotypic response. In various embodiments, the method comprises activating the second cell population to create an activated cell population. In various embodiments, the method comprises transducing the activated cell population to create a final cell population. In various embodiments, the method comprises harvesting a final cell population to generate a cell therapy product, wherein the subsequent phenotypic response comprises an increase of central memory or naïve cells in the final cell population.

### FIGURES

FIG. 1 illustrates steps in a method for a T cell manufacturing process according to various embodiments.

FIG. 2 illustrates a peristaltic pump in three operational positions according to various embodiments.

FIG. 3 illustrates a mechanical force generating system including a flexible housing, a pump, a sampling port and fluid channel according to various embodiments.

FIG. 4 shows experimental data for T cells being subjected to shear stress over various times in initial experiments for pump selection and operational parameter assessment.

FIG. 5 illustrates a study design for peristaltic pump shearing used in the examples detailed herein.

FIG. 6 shows experimental data for early T cell activation.

FIG. 7 shows experimental data for late T cell activation.

FIG. 8 shows experimental data for net change in CD3+CD4+ T cell phenotype post peristaltic pump and at harvest for fresh T cells and PBMCs.

FIG. 9 shows experimental data for net change in CD3+CD4+ T cell phenotype post peristaltic pump and at harvest for cryo preserved T cells and PBMCs.

FIG. 10 shows experimental data for net change in CD3+CD8+ T cell phenotype post peristaltic pump and at harvest for fresh T cells and PBMCs.

FIG. 11 shows experimental data for net change in CD3+CD8+ T cell phenotype post peristaltic pump and at harvest for cryo preserved T cells and PBMCs.

FIG. 12 shows experimental data for T cell CD4+/CD8+ ratio.

FIG. 13 shows experimental data for programmed cell death protein 1 marker CD3+PD-1+.

FIG. 14 shows experimental data for co-stimulatory marker CD3+CD27+.

FIG. 15 shows experimental data for co-stimulatory marker CD3+CD28+.

FIG. 16 shows experimental data for T cell, B cell, and NK cell populations.

FIG. 17 shows experimental data for net IL-2 concentration change.

FIG. 18 illustrates a method of applying a shear force to a first cell population to cause a subsequent phenotypic response according to various embodiments.

### DETAILED DESCRIPTION

### I. Definitions

In order that the present disclosure may be more readily understood, certain terms are first defined. As used in this application, except as otherwise expressly provided herein, each of the following terms shall have the meaning set forth below. Additional definitions are set forth throughout the application. The headings provided herein are not limitations of the various aspects of the disclosure, which aspects should be understood by reference to the specification as a whole.

As used herein, the terms “a” and “an” are used per standard convention and mean one or more, unless context dictates otherwise.

As used herein, the term “and/or” may be understood as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or,” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term the use of the alternative (e.g., “or”) should be understood to mean either one, both, or any combination thereof of the alternatives.

As used herein, the term “about,” “around,” or “approximately” may refer to a value or composition that is within an acceptable error range for the value or composition as determined by one of ordinary skill in the art, which may depend in part on how the value or composition is measured or determined, i.e., the limitations of the measurement system. When values or compositions are provided in the instant disclosure, unless otherwise stated, the meaning of “about” should be assumed to be within an acceptable error range for that value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Throughout the specification the word “comprising,” or variations such as “comprises” or “comprising,” may be understood to imply the inclusion of a stated element, integer or step, or group of elements, integers or steps, but not the exclusion of any other element, integer or step, or group of elements, integers or steps.

As used herein, the term “administering” may refer to the physical introduction of an agent to a subject, such as a modified T cell and/or a CD38 therapy as disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” may means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In various embodiments, the formulation is administered via a non-parenteral route, e.g., orally. Other non-parenteral routes include a topical, epidermal or mucosal route of administration, for example, intranasally, vaginally, rectally, sublingually or topically. Administering may also be performed, for example, once, a plurality of times, and/or over one or more extended periods.

As used herein, the term “antibody” (Ab) may include, without limitation, a glycoprotein immunoglobulin which binds specifically to an antigen. Antibodies described in the present application may include antibodies used for quantifying process improvements in a T cell manufacturing process. Various parts of antibodies may be analogous to other compounds or parts of compounds (e.g., T cell receptors). In general, an antibody may comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region may comprise one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In general, human antibodies may be approximately 150 kD tetrameric agents comprised of two identical heavy (H) chain polypeptides (about 50 kD each) and two identical light (L) chain polypeptides (about 25 kD each) that associate with each other into what is commonly referred to as a “Y-shaped” structure. The heavy and light chains may be linked or connected to one another by a single disulfide bond; two other disulfide bonds connect the heavy chain hinge regions to one another, so that the dimers are connected to one another and the tetramer is formed. Naturally produced antibodies are also glycosylated, e.g., on the C_{H}2 domain.

As used herein, the term “human antibody” may refer to antibodies having variable and constant domain sequences generated, assembled, or derived from human immunoglobulin sequences, or sequences indistinguishable therefrom in accordance with various embodiments. In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation). The term “humanized” is intended to comprise antibodies having a variable domain with a sequence derived from a variable domain of a non-human species (e.g., a mouse), modified to be more similar to a human germline encoded sequence. In various embodiments, a “humanized” antibody comprises one or more framework domains having substantially the amino acid sequence of a human framework domain, and one or more complementary determining regions having substantially the amino acid sequence as that of a non-human antibody. In various embodiments, a humanized antibody comprises at least a portion of an immunoglobulin constant region (Fc), generally that of a human immunoglobulin constant domain. In some embodiments, a humanized antibodies may comprise a C_{H}1, hinge, C_{H}2, C_{H}3, and, optionally, a C_{H}4 region of a human heavy chain constant domain.

Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen binding fragments of any of the above. In various embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof, and human heavy-chain antibodies (UniAbs)), camelid antibodies, single chain or Tandem diabodies (TandAb®), Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s.

An immunoglobulin may be derived from any of the commonly known isotypes, including but not limited to IgA, secretory IgA, IgG, IgE and IgM. IgG subclasses are also well known to those in the art and include but are not limited to human IgG1, IgG2, IgG3 and IgG4. “Isotype” may refer to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

As used herein, the terms “antigen binding molecule,” “antigen binding portion,” “antigen binding fragment,” “antibody fragment” or “antigen binding domain” may refer to any molecule that comprises the antigen binding parts of the molecule. In an example an antigen binding molecule may be an antibody, or portion thereof, such as an scFv. In various examples an antigen biding molecule may be a portion of a TCR that binds an antigen and may be the antigen binding portion of the TCR alpha chain and/or the antigen binding portion of a TCR alpha chain. Non-limiting examples of antigen binding molecule targets may include CD20, CD19, CLL-1, BCMA, EGFR, HER2, GPC3, GD2, or any combination thereof. In an example, an antigen biding molecule may be a portion of NKG2D that binds an NKG2D ligand. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are additional non-limiting examples of suitable antigen binding molecules. In some embodiments, the antigen binding molecule binds to an antigen on a tumor cell. In some embodiments, the antigen binding molecule may bind to an antigen on a cell involved in a hyperproliferative disease or to a viral or bacterial antigen. In various embodiments, an antigen binding molecule may include a chimeric antigen receptor (CAR) or an engineered T cell receptor (TCR). In various embodiments, the antigen binding molecule or domain may include an antibody fragment that may specifically bind to the antigen, including one or more of the complementarity determining regions (CDRs) thereof. In various embodiments, the antigen binding molecule may include a single chain variable fragment (scFv).

In various embodiments, an antigen binding molecule may comprise an antibody memetics. Antibody memetics may include compounds similar to antibodies capable of binding to antigens. In various embodiments, antibody memetics may not be structurally related to antibodies. In various embodiments, antibody mimetics may include an artificial peptide or protein. In various embodiments, an antibody mimetic may include one or more nucleic acids. In various embodiments, a molar mass of an antibody memetic may range from about 3 to 20 kDa. Non-limiting examples of memetics may include affibody molecules, affilins, affimers, affitins, alphabodies, anticalins, avimers, DARPins, fynomers, gastrobodies, kunitz domain peptides, monobodies, nanoCLAMPs, optimers, repebodies, pronectin, centyrins, and obodies. In various circumstances, antibody mimetics may improve solubility, tissue penetration, and stability when exposed to heat and enzymes.

In various instances, a CDR may be substantially identical to one found in a reference antibody (e.g., an antibody of the present disclosure) and/or the sequence of a CDR provided in the present disclosure. In some embodiments, a CDR is substantially identical to a reference CDR in that it is either identical in sequence or contains between 1, 2, 3, 4, or 5 (e.g., 1-5) amino acid substitutions as compared with the reference CDR. In some embodiments a CDR may be substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In various embodiments a CDR is substantially identical to a reference CDR in that it shows at least 96%, 96%, 97%, 98%, 99%, or 100% sequence identity with the reference CDR. In various embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g., 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody. In various embodiments, an antigen binding fragment cross-competes with the reference antibody, for example, binding to substantially the same or identical epitope as the reference antibody.

An antigen binding fragment may be produced by any means. For example, in various embodiments, an antigen binding fragment may be enzymatically or chemically produced by fragmentation of an intact antibody. In various embodiments, an antigen binding fragment may be recombinantly produced (such as by expression of an engineered nucleic acid sequence). In various embodiments, an antigen binding fragment may be wholly or partially synthetically produced. In various embodiments, an antigen binding fragment may have a length of at least about 50, 60, 70, 80, 90, 100, 110, 120, 130, 140, 150, 160, 170, 180, 190 amino acids or more; in various embodiments at least about 200 amino acids (e.g., 50-100, 50-150, 50-200, or 100-200 amino acids).

As used herein, the terms “variable region” or “variable domain” are used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In various embodiments, the variable region may be a human variable region. In various embodiments, the variable region comprises rodent or murine CDRs and human framework regions (FRs). In various embodiments, the variable region may be a primate (e.g., non-human primate) variable region. In various embodiments, the variable region comprises rodent or murine CDRs and primate (e.g., non-human primate) framework regions (FRs).

As used herein, the terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

As used herein, the terms “constant region” and “constant domain” are interchangeable and have a meaning common in the art. The constant region may be an antibody portion, e.g., a carboxyl terminal portion of a light and/or heavy chain which is not directly involved in binding of an antibody to antigen, but which can exhibit various effector functions, such as interaction with the Fc receptor. The constant region of an immunoglobulin molecule generally has a more conserved amino acid sequence relative to an immunoglobulin variable domain.

As used herein, the terms “heavy chain” when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

As used herein, the terms “light chain” when used in reference to an antibody may refer to any distinct type, e.g., kappa (κ) or lambda (λ) based on the amino acid sequence of the constant domains. Light chain amino acid sequences are well known in the art. In various embodiments, the light chain may be a human light chain.

An “antigen” refers to a compound, composition, or substance that may stimulate the production of antibodies or a T cell response in a human or animal, including compositions (such as one that includes a tumor-specific protein) that are injected or absorbed into a human or animal. An antigen reacts with the products of specific humoral or cellular immunity, including those induced by heterologous antigens, such as the disclosed antigens. A “target antigen” or “target antigen of interest” is an antigen that is not substantially found on the surface of other normal (desired) cells and to which a binding domain of a TCR or CAR contemplated herein, is designed to bind. A person of skill in the art would readily understand that any macromolecule, including virtually all proteins or peptides, can serve as an antigen. An antigen can be endogenously expressed, i.e. expressed by genomic DNA, or can be recombinantly expressed.

An antigen can be specific to a certain tissue, such as a cancer cell, or it can be broadly expressed. In addition, fragments of larger molecules can act as antigens. A “target” is any molecule bound by a binding motif, CAR, TCR or antigen binding agent, e.g., an antibody.

As used herein, the term “antigen-specific targeting region” (ASTR) may refer to the region of the CAR or TCR which targets specific antigens. In various embodiments, the targeting regions on the CAR or TCR may be extracellular. In various embodiments, the antigen-specific targeting regions comprise an antibody or a functional equivalent thereof or a fragment thereof or a derivative thereof and each of the targeting regions target a different antigen. In various embodiments, the targeting regions may comprise full length heavy chain, Fab fragments, single chain Fv (scFv) fragments, divalent single chain antibodies or diabodies, each of which are specific to the target antigen. There are, however, numerous alternatives, such as linked cytokines (which leads to recognition of cells bearing the cytokine receptor), affibodies, ligand binding domains from naturally occurring receptors soluble protein/peptide ligand for a receptor (for example on a tumor cell), peptides, and vaccines to prompt an immune response, which may each be used in various embodiments of this disclosure. In fact, almost any molecule that binds a given antigen with high affinity can be used as an antigen-specific targeting region, as will be appreciated by those of skill in the art.

As used herein, the term “apheresis” or “apheresis material” may originate from blood. In various embodiments, patient/donor derived blood may undergo an apheresis process to generate an apheresis material. In various embodiments, the apheresis material may include leukocytes. Additional examples of an apheresis material may include platelets, white blood cells, or any constituent of blood. In more specific examples, T cells may be collected from patients for later processing (e.g., T cell modification). In various embodiments, apheresis may be used as a starting material in a cell therapy manufacturing process.

In various embodiments, an apheresis process may include collecting whole blood from a patient. In various embodiments, an apheresis process may be an initial part of a T cell manufacturing process. In various embodiments, the whole blood may be subjected to a size separation protocol. A common size selection protocol may employ use of centrifuge to separate the whole blood components by their respective weights. For example, this technique may result in generation of separation layers including plasma, a buffy coat, and erythrocytes. In various embodiments, the resulting plasma layer may make up about 55% of the total volume. In various embodiments, the resulting erythrocyte layer may make up about 45% of the total volume. In various embodiments, the resulting buffy coat layer may make up about less than 1% of the total volume. In various embodiments, the buffy coat may include leukocytes and platelets which may be further processed to further separate components of the buffy coat. For example, T cells may be isolated for later modification using the techniques described herein and elsewhere.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient. In various embodiments, the methods described herein may be incorporated into an autologous T cell manufacturing system and/or process.

The term “allogeneic” refers to any material derived from one individual which is then introduced to another individual of the same species, e.g., allogeneic T cell transplantation. In various embodiments, the methods described herein may be incorporated into an allogeneic T cell manufacturing system and/or process.

As used herein, the term “binding” generally refers to a non-covalent association between or among two or more entities. Direct binding may involve physical contact between entities or moieties. “Indirect” binding involves physical interaction by way of physical contact with one or more intermediate entities. Binding between two or more entities may be assessed in any of a variety of contexts, e.g., where interacting entities or moieties are studied in isolation or in the context of more complex systems (e.g., while covalently or otherwise associated with a carrier entity and/or in a biological system such as a cell).

The term “cancer” may refer to a broad group of various diseases characterized by the uncontrolled growth of abnormal cells in the body. The cell therapy products of the T cell manufacturing processes described herein are designed to generate treatments for cancer among other things. Cancer may mean unregulated cell division and growth results in the formation of malignant tumors that invade neighboring tissues and may also metastasize to distant parts of the body through the lymphatic system or bloodstream. A “cancer” or “cancer tissue” may include a tumor. In this application, the term cancer may be synonymous with malignancy. Examples of cancers that may be treated by the methods or cell therapy products generated by the methods herein include, but are not limited to, cancers of the immune system including lymphoma, leukemia, myeloma, and other leukocyte malignancies. In various embodiments, the methods or products derived from the methods disclosed herein may be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T cell lymphoma, environmentally induced cancers including those induced by asbestos, other B cell malignancies, and combinations of said cancers. In various embodiments, the cancer is multiple myeloma. In various embodiments, the cancer may be NHL. The particular cancer may be responsive to chemo- or radiation therapy or the cancer may be refractory. A refractory cancer refers to a cancer that is not amenable to surgical intervention and the cancer is either initially unresponsive to chemo- or radiation therapy or the cancer becomes unresponsive over time.

As used herein, the term “chimeric antigen receptor” or “CAR” may refer to a molecule engineered to comprise a binding domain and a means of activating immune cells (for example T cells such as naive T cells, central memory T cells, effector memory T cells, NK cells or combination thereof) upon antigen binding. CARs are also known as artificial T cell receptors, chimeric T cell receptors or chimeric immunoreceptors. In various embodiments, a CAR may comprise a binding domain, an extracellular domain, a transmembrane domain, one or more co-stimulatory domains, and an intracellular signaling domain. A T cell that has been genetically engineered to express a chimeric antigen receptor may be referred to as a CAR T cell. Similarly, an NK cell that has been genetically engineered to express a chimeric antigen receptor may be referred to as a CAR NK cell.

In various embodiments, the binding domain of a CAR may be followed by a “spacer,” or, “hinge,” which may refer to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region may be an immunoglobulin hinge region and may be a wild type immunoglobulin hinge region or an altered wild type immunoglobulin hinge region, such as an IgG4 hinge. Other exemplary hinge regions used in the CARs described herein include the hinge region derived from the extracellular regions of type 1 membrane proteins such as CD8alpha, CD4, CD28 and CD7, which may be wild-type hinge regions from these molecules or may be altered.

As used herein, the term “cell therapy product” may refer to a therapy including viable cells. The viable cells may be administered to a patient. Administration may occur by injection, translation, or infusion. In various embodiments, the viable cells may include immune cells. In various embodiments, the immune cells may include T cells. In various embodiments, the T cells may include chimeric antigen receptors (CARs).

As used herein, the terms “costimulatory signaling domain” and “costimulatory domain” are used interchangeably and may refer to the portion of the CAR comprising the intracellular domain of a costimulatory molecule. Costimulatory molecules are cell surface molecules other than antigen receptors or Fc receptors that provide a second signal required for efficient activation and function of T lymphocytes upon binding to antigen. Non-limiting examples of such co-stimulatory molecules may include CD27, CD28, 4-1 BB (CD137), OX40 (CD134), CD30, CD40, PD-1, ICOS (CD278), LFA-1, CD2, CD7, LIGHT, NKD2C, 2B4, CD137, DAP12, B7-H2 and a ligand that specifically binds CD83. A skilled artisan will appreciate that other costimulatory domains may be used with the CARs described herein. In various embodiments, the inclusion of one or more costimulatory signaling domains may enhance the efficacy and expansion of T cells and NK cells expressing CAR receptors. In various embodiments, he intracellular signaling and costimulatory signaling domains may be linked in any order in tandem to the carboxyl terminus of the transmembrane domain.

Although CARs engineered to comprise a signaling domain from CD3 or FcRgamma have been shown to deliver a potent signal for T cell activation and effector function, they are not sufficient to elicit signals that promote T cell survival and expansion in the absence of a concomitant costimulatory signal. Other CARs containing a binding domain, a hinge, a transmembrane and the signaling domain derived from CD3zeta or FcRgamma together with one or more costimulatory signaling domains (e.g., intracellular costimulatory domains derived from 4-1BB, CD28, CD134 and CD278) may more effectively direct antitumor activity as well as increased cytokine secretion, lytic activity, survival and proliferation in CAR expressing T cells in vitro, and in animal models and cancer patients (Milone et al., Molecular Therapy, 2009; 17: 1453-1464; Zhong et al., Molecular Therapy, 2010; 18: 413-420; Carpenito et al., PNAS, 2009; 106:3360-3365).

A “costimulatory signal” may refer to a signal, which in combination with a primary signal, such as TCR/CD3 ligation, leads to a T cell response, such as, but not limited to, proliferation and/or upregulation or down regulation of key molecules.

A “costimulatory ligand” includes a molecule on an antigen presenting cell that specifically binds a cognate co-stimulatory molecule on a T cell. Binding of the costimulatory ligand provides a signal that mediates a T cell response, including, but not limited to, proliferation, activation, differentiation, and the like. A costimulatory ligand induces a signal that is in addition to the primary signal provided by a stimulatory molecule, for instance, by binding of a T cell receptor (TCR)/CD3 complex with a major histocompatibility complex (MHC) molecule loaded with peptide. A co-stimulatory ligand can include, but is not limited to, 3TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

As used herein, the term “costimulatory molecule” may refer to a cognate binding partner on a T cell that specifically binds with a costimulatory ligand, thereby mediating a costimulatory response by the T cell, such as, but not limited to, proliferation. Costimulatory molecules include, but are not limited to, A “costimulatory molecule” is a cognate binding partner on a T cell that specifically binds with a costimulatory ligand, thereby mediating a costimulatory response by the T cell, such as, but not limited to, proliferation. Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CD1-1a, CD1-1b, CD1-1c, CD1-1d, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDl 1a/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Ly108), SLAMF7, SLP-76, TNF, TNFr, TNFR2, Toll ligand receptor, TRANCE/RANKL, VLA1, or VLA-6, or fragments, truncations, or combinations thereof.

As used herein, the term “extracellular domain” (or “ECD”) may refer to a portion of a polypeptide that, when the polypeptide is present in a cell membrane, is understood to reside outside of the cell membrane, in the extracellular space. Ecto domain may be used herein interchangeably with extracellular domain.

As used herein, the term “extracellular ligand-binding domain” may refer to an oligo- or polypeptide that is capable of binding a ligand, e.g., a cell surface molecule. For example, an extracellular ligand-binding domain may be selected to recognize a ligand that acts as a cell surface marker on target cells associated with a particular disease state (e.g., cancer). Non-limiting examples of cell surface markers that may act as ligands include those associated with viral, bacterial and parasitic infections, autoimmune disease and cancer cells.

The term “genetically engineered” or “engineered” may refer to a method of modifying the genome of a cell, including, but not limited to, deleting a coding or non-coding region or a portion thereof or inserting a coding region or a portion thereof. In various embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

As used herein, the term “immunotherapy” may refer to the treatment of a subject afflicted with, or at risk of contracting or suffering a recurrence of, a disease by a method comprising inducing, enhancing, suppressing or otherwise modifying an immune response.

Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT™), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. Nos. 7,741,465, 6,319,494, 5,728,388, and International Publication No. WO 2008/081035. In various embodiments, the immunotherapy comprises CAR T cell treatment. In various embodiments, the methods for upregulating low-density lipoprotein receptor (LDL-R) expression described herein may be incorporated into any of the immunotherapy workflows described herein and elsewhere.

T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

As used herein, the terms “intracellular signaling domain” and “signaling domain” are used interchangeably and may refer to the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain. As used herein, the term “effector function” may refer to a specialized function of the cell. Effector function of the T cell, for example, may be cytolytic activity or help or activity including the secretion of a cytokine.

Thus, the terms “intracellular signaling domain or “signaling domain,” used interchangeably herein, refer to the portion of a protein which transduces the effector function signal and that directs the cell to perform a specialized function. While usually the entire intracellular signaling domain may be employed, in many cases it is not necessary to use the entire domain. To the extent that a truncated portion of an intracellular signaling domain is used, such truncated portion may be used in place of the entire domain as long as it transduces the effector function signal. The term intracellular signaling domain may include any truncated portion of the intracellular signaling domain sufficient to transducing effector function signal. The intracellular signaling domain is also known as the, “signal transduction domain,” and is typically derived from portions of the human CD3 or FcRy chains.

It is known that signals generated through the T cell receptor alone are insufficient for full activation of the T cell and that a secondary, or costimulatory signal is also required. Thus, T cell activation can be said to be mediated by two distinct classes of cytoplasmic signaling sequences: those that initiate antigen dependent primary activation through the T cell receptor (primary cytoplasmic signaling sequences) and those that act in an antigen independent manner to provide a secondary or costimulatory signal (secondary cytoplasmic signaling sequences). Cytoplasmic signaling sequences that act in a costimulatory manner may contain signaling domains which are known as immunoreceptor tyrosine-based activation domain or ITAMs.

Non-limiting examples of ITAM containing primary cytoplasmic signaling sequences that may be of particular use in the embodiments described herein may include those derived from DAP10, DAP12, TCRzeta, FcRgamma, FcRbeta, CD3zeta, CD3gamma, CD3delta, CD3epsilon, CD5, CD22, CD79a, CD79b and CD66d.

The term “lymphocyte” as used herein includes natural killer (NK) cells, T cells, or B cells. NK cells are a type of cytotoxic (cell toxic) lymphocyte that represent a major component of the innate immune system. In various embodiments, the technologies described herein may be applicable to lymphocytes. NK cells reject tumors and cells infected by viruses. NK cells reject tumor cells through the process of apoptosis or programmed cell death. They were termed “natural killers” because they do not require activation in order to kill cells. T cells play a major role in cell-mediated-immunity (no antibody involvement). Its T cell receptors (TCR) differentiate themselves from other lymphocyte types. The thymus, a specialized organ of the immune system, is primarily responsible for the T cell's maturation. T cells can be divided in CD4+ subset (i.e. Helper T cells) and CD8+ subset (i.e. Cytotoxic T cells). Based on the level of differentiation, T cells can also be divided into 4 subsets: naïve, CM (central memory), EM (effector memory) and TEMRA (Terminally differentiated effector memory) B-cells, on the other hand, play a principal role in humoral immunity (i.e. with antibody involvement). B-cells makes antibodies and antigens and perform the role of antigen-presenting cells (APCs) and turns into memory B-cells after activation by antigen interaction. In mammals, immature B-cells are formed in the bone marrow, from where its name is derived.

As used herein, the term “patient” means any human who is being treated for an abnormal physiological condition, such as cancer or has been formally diagnosed with a disorder, those without formally recognized disorders, those receiving medical attention, those at risk of developing the disorders, etc. The terms “subject” and “patient” are used interchangeably herein and include both human and non-human animal subjects.

As used herein, the term “selected T cells” may refer to T cells that have undergone a T cell selection process. In various embodiments, selected T cells may refer to selected T cells that have been cryo preserved and may be made available for further processing after undergoing a thawing protocol. In various embodiments, selected T cells may remain fresh and never have gone through a cryo preservation process.

As used herein, the term “sample” may generally refer to an aliquot of material obtained or derived from a source of interest. In various embodiments, a sample may be a starting material. In various embodiments, a sample or a source of interest may include an apheresis material. In various embodiments, a source of interest is a biological or environmental source. In various embodiments, a source of interest may comprise a cell or an organism, such as a cell population, tissue, or animal (e.g., a human). In various embodiments, a source of interest may include biological tissue or fluid. In various embodiments, a biological tissue or fluid may comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In various embodiments, a biological fluid may comprise an intracellular fluid, an extracellular fluid, an intravascular fluid (blood plasma), an interstitial fluid, a lymphatic fluid, and/or a transcellular fluid. In various embodiments, a biological fluid may comprise a plant exudate. In various embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In various embodiments, a biological sample may comprise cells obtained from an individual. In various embodiments, a sample may include a “primary sample” obtained directly from a source of interest by any appropriate means. In various embodiments, as will be clear from context, the term “sample” may refer to a preparation that is obtained by processing (e.g., by removing one or more components of and/or by adding one or more agents to) a primary sample. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

As used herein, the term “single chain variable fragment,” “single-chain antibody variable fragments,” or “scFv” antibodies refer to forms of antibodies comprising the variable regions of only the heavy and light chains, connected by a linker peptide.

As used herein, the term “stimulation,” may refer to a primary response induced by binding of a stimulatory molecule with its cognate ligand, wherein the binding mediates a signal transduction event. A “stimulatory molecule” may include a molecule on a T cell, e.g., the T cell receptor (TCR)/CD3 complex, that specifically binds with a cognate stimulatory ligand present on an antigen present cell. A “stimulatory ligand” may include a ligand that when present on an antigen presenting cell (e.g., an APC, a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an anti-CD3 antibody (such as OKT3), an MHC Class I molecule loaded with a peptide, a superagonist anti-CD2 antibody, and a superagonist anti-CD28 antibody.

As used herein, the terms “T cell receptor” and “TCR” are used interchangeably and may refer to antigen-recognition molecules present on the surface of T cells. During normal T cell development, each of the four TCR genes, α, β, γ, and δ, may rearrange leading to highly diverse TCR proteins. Examples of TCR based T cell therapies are disclosed in International Patent Application Nos. PCT/US2013/059608 and PCT/US2015/033129, which are hereby incorporated herein by reference in their entirety.

As used herein, the term “T cell exhaustion” may refer to a state T cells can enter during chronic stimulation. T cell exhaustion may lead to loss of effector activity, loss of proliferation capacity, and other dysfunctions. The methods described herein may reduce T cell exhaustion by eliminating the need for a T cell activation step in a T cell manufacturing process.

The terms “transduction” and “transduced” may refer to the process whereby foreign DNA is introduced into a cell via viral vector (see Hartd and Jones (1997) “Genetics: Principles and Analysis.” 4^{th} ed, Jones & Bartlett). In various embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

As used herein, the term “transmembrane domain” may refer to a domain of a polypeptide that includes at least one contiguous amino acid sequence that traverses a lipid bilayer when present in the corresponding endogenous polypeptide when expressed in a mammalian cell. For example, a transmembrane domain can include one, two, three, four, five, six, seven, eight, nine, or ten contiguous amino acid sequences that each traverse a lipid bilayer when present in the corresponding endogenous polypeptide when expressed in a mammalian cell. In various embodiments, a transmembrane domain may include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In various embodiments, a transmembrane domain may include two or more contiguous amino acid sequences (that each traverse a lipid bilayer when present in the corresponding endogenous polypeptide when expressed in a mammalian cell) that form a β-barrel secondary structure in the lipid bilayer. In various embodiments, the transmembrane region or domain may include the portion of a CAR that anchors the extracellular binding portion to the plasma membrane of the immune effector cell and facilitates binding of the binding domain to the target antigen. The transmembrane domain may include a CD3zeta transmembrane domain, however other transmembrane domains that may be employed include those obtained from CD8alpha, CD4, CD28, CD45, CD9, CD16, CD22, CD33, CD64, CD80, CD86, CD134, CD137, NKG2D, 2B4 and CD154. In various embodiments, the transmembrane domain may be synthetic in which case it would comprise predominantly hydrophobic residues such as leucine and valine.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In various embodiments, “treatment” or “treating” includes a partial remission. In another embodiment, “treatment” or “treating” includes a complete remission.

As used herein, the term “vector” may refer to a recipient nucleic acid molecule modified to comprise or incorporate a provided nucleic acid sequence. One type of vector may be a “plasmid,” which may refer to a circular double stranded DNA molecule into which additional DNA may be ligated. Another type of vector may be a viral vector, wherein additional DNA segments may be ligated into the viral genome. Certain vectors may be capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

### II. Overview

Currently existing equipment used in the cell therapy industry may be adapted from applications such as stem cell research and the near mature biologics industry, but often are not designed or optimized for T cell or CAR T cell manufacturing. Equipment development and optimization may not be a priority for pharmaceutical companies that are attempting to receive regulatory approval for their product. This is often because equipment and methods may have been previously validated and switching to technology designed and optimized for the purposes described herein may require additional regulatory approval.

As described herein, human immune cells (e.g., T cells) may be sensitive to environmental conditions. As such, use of equipment that does not take environmental conditions such as those found in the human body poses challenge. For example, equipment used in the biologics technology space, such as upright stirred tank bioreactors, may be unsuitable or suboptimal when adapted for T cell and/or CAR T cell manufacturing. The technologies described herein may enable specific application of mechanical forces (e.g., shear stress) to cells, thereby, enabling robust activation, transduction, and expansion. Further, the technologies herein enable better phenotypic outcomes in a cell therapy product such as increased naïve and central memory cells. The systems and methods described herein may be designed and optimized for T cell and CAR T cell manufacturing. Further, use of the technologies described herein may be suitable and or optimized for immune cells. Even further, use of the technologies described herein may be suitable or optimized for eukaryotic cells (e.g., human cells).

Optimizing T cell and/or CAR-T cell processes and equipment (e.g., the systems and methods described herein) to mimic the microenvironment found in the human body may result in advantages such as a more robust T cell activation, transduction, and expansion. Human immune cells may be subject to similar shear stresses found in the human body and have more naïve cell phenotypes which may lead to better clinical responses (e.g., efficacy and improved safety profiles). Understanding the effects and mechanisms of shear stress on human immune cells that are commonly associated with CAR-T cell therapies, such as purified T cells and peripheral blood mononuclear cells (PBMCs), may allow development of cell therapy specific equipment. Exploiting these target cells by inducing additional shear stress can also result in process advantages.

Mechanical force (e.g., shear stress) may be applied using a variety of different equipment types such as peristaltic pumps and centrifugation, in accordance with various embodiments. The technology described herein opposes the widely accepted industry view that excessive shear stress may be detrimental to cell cultures. Exposing T cells and PBMCs to an ideal amount of shear stress (e.g., a total accumulated shear force) using the technologies described herein may yield favorable results. Various embodiments comprise use of peristaltic pumps generating specified flow rates for optimized durations as a source of shear stress.

Various embodiments comprise use of centrifuges to generate optimized amounts shear stress at optimized durations as a source of shear stress. In various embodiments, a combination of peristaltic pumping and centrifugation may yield optimal shear forces for a cell therapy manufacturing process. The present disclosure, among other things, provides insights and technologies useful in applying mechanical forces to immune cells to improve activation, transduction, and expansion steps in a manufacturing process. More specifically, the embodiments described herein may be useful in the life sciences and pharmaceutical industries to ensure better cell therapy performance through improvements in a manufacturing process.

Embodiments of systems, apparatuses, and methods for applying a mechanical force to a population of cells during a cell therapy manufacturing process are described in the accompanying description and figures. In the figures, numerous specific details are set forth to provide a thorough understanding of certain embodiments. A skilled artisan will appreciate that the systems and methods described herein may be used in a variety of ways and circumstances that are not limited to what is specifically detailed. Additionally, the skilled artisan will appreciate that certain embodiments may be practiced without these specific details. Furthermore, one skilled in the art can readily appreciate that the specific sequences in which methods are presented and performed are illustrative and it is contemplated that the sequences can be varied and still remain within the spirit and scope of the embodiments.

While the present teachings are described in conjunction with various embodiments, it is not intended that the present teachings be limited to such embodiments. On the contrary, the present teachings encompass various alternatives, modifications, and equivalents, as will be appreciated by those skilled in the art.

Technologies enabling application of mechanical forces (e.g., shear stress) on human immune cells that are commonly associated with CAR-T cell therapies, such as purified T cells and peripheral blood mononuclear cells (PBMCs) are described herein.

### III. Exemplary T Cell Manufacturing Process

Provided herein are systems and methods for manufacturing engineered human lymphocytes (e.g., T cells, CAR T cells, etc.) for cell therapy products according to various embodiments. Exemplary hardware may be described herein and elsewhere for carrying out the steps in accordance with various embodiments. Lymphocytes for immunotherapy may come from any source known in the art. For example, T cells may be differentiated in vitro from a hematopoietic stem cell population, or T cells may be obtained from a subject according to various embodiments. T cells may be obtained from peripheral blood mononuclear cells (PBMCs), bone marrow, lymph node tissue, cord blood, thymus tissue, tissue from a site of infection, ascites, pleural effusion, spleen tissue, and/or tumors depending on the application and the various embodiments. In addition, the T cells may be derived from one or more T cell lines available in the art. T cells may also be obtained from a unit of blood collected from a subject using any number of techniques known to the skilled artisan, such as apheresis. Additional methods of isolating T cells for a T cell therapy are disclosed in U.S. Patent Publication No. US20130287748A1, which is hereby incorporated by reference in its entirety and for all purposes.

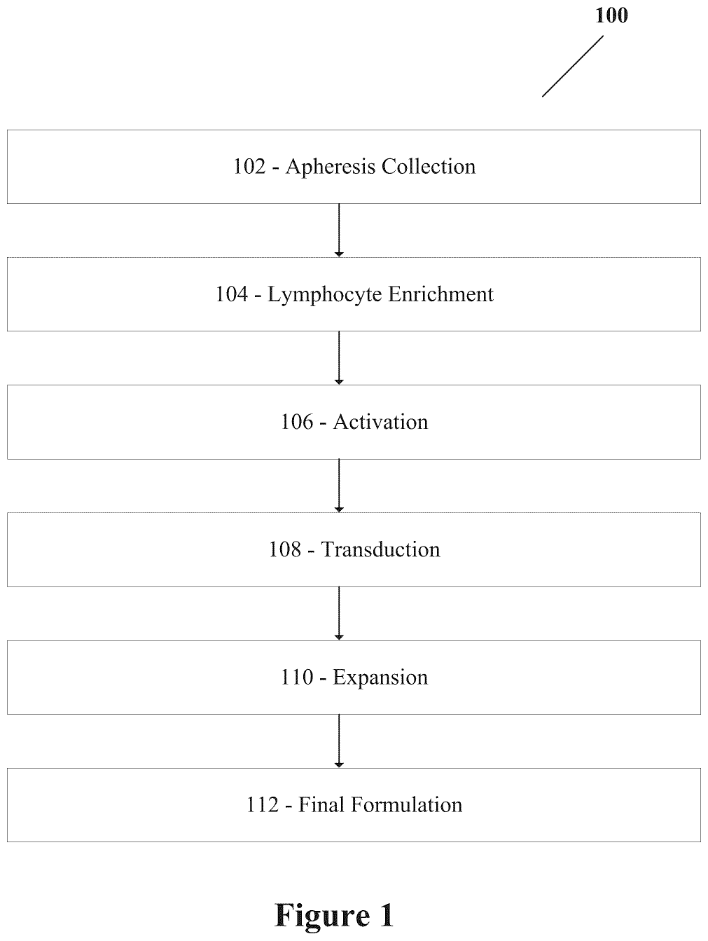

FIG. 1 illustrates steps in a method for a T cell manufacturing process 100 according to various embodiments. In various embodiments, one or more of the steps described herein may be optional. For example, in various embodiments an activation step may not be necessary as described in “Systems and Methods for Upregulating Low-Density Lipoprotein Receptor (LDL-R) Expression”; PCT/US2024/061010, the disclosure of which is hereby incorporated by reference in its entirety for all purposes. Conversely, in various embodiments, the manufacturing process 100 may comprise steps not described herein or specifically named in subsections (e.g., wash steps, addition of reagents, etc.). Further, the steps described in the manufacturing process 100 may be completed in a different order than described herein according to various embodiments. Even further, the two or more of the steps described herein may be combined into a single step and/or be performed concurrently according to various embodiments. Additionally, many steps may be incorporated into the workflow described herein that may not be specifically disclosed herein.

The steps in the T cell manufacturing process 100 may include one or more an apheresis collection 102, a lymphocyte enrichment 104, an activation 106, a transduction 108, an expansion 110, and a final formulation according to various embodiments. A skilled artisan will appreciate that a T cell manufacturing process 100 may include one or more additional sub steps (e.g., selection), may include fewer steps, repeated steps, and/or reordered steps depending on the cell type, application, or any number of other factors.

a. Apheresis Collection

In various embodiments, the manufacturing method 100 may include apheresis collection 102 as an initial step. In various embodiments, apheresis collection 102 may be a step in a manufacturing process for autologous T cell therapies, as described in the patent application US20150344844A1, the disclosure of which is hereby incorporated by reference in its entirety for all purposes. In various embodiments, apheresis collection 102 may be a step in a manufacturing process for allogenic T cell therapies.

In various embodiments, apheresis collection 102 may comprise extraction of white blood cells (e.g., lymphocytes) from a patient or donor. In various embodiments, commercial systems may be incorporated into any of the workflows described herein such as the COBE® Spectra, Spectra Optia®, or Fenwal™ Amicus®.

In various embodiments, apheresis collection 102 may comprise a step of drawing blood from a patient and the blood may then be passed through a machine that may separate the desired white blood cells from other blood components such as red blood cells and plasma. In various embodiments, the other blood components may then be returned to the patient's circulatory system.

In various embodiments, an apheresis collection 102 step may yield approximately 200 to 400 milliliters of a cell therapy product. In various embodiments, the cell therapy product may include a rich population of peripheral blood mononuclear cells (PBMCs), including T cells.

In various embodiments, apheresis collection 102 may be performed at a clinical site. In various embodiments, the apheresis may be shipped under controlled conditions (typically at 1-10° C.) to a manufacturing facility for further processing. In various embodiments, systems and methods for shipping apheresis described in WO2025034514A1 may be used for transportation of the apheresis, the disclosure of which is hereby incorporated by reference in its entirety for all purposes.

In various embodiments, the cell therapy product (e.g., the collected cells) may then be subjected to volume reduction and lymphocyte enrichment steps to prepare them for activation, genetic modification, and/or expansion.

b. Lymphocyte Enrichment

In various embodiments, the manufacturing process 100 may include lymphocyte enrichment 104. In various embodiments, lymphocyte enrichment 104 may be a step in the preparation of autologous T cells for cell therapy. In various embodiments, lymphocyte enrichment 104 may be a step in the preparation of allogenic T cells for cell therapy. In various embodiments, lymphocyte enrichment 104 may follow apheresis collection 102 and the optional step of volume reduction.

In various embodiments, cells of interest may be isolated via positive or negative selection using density gradient, magnetic bead, and/or acoustic forces to obtain a mass of enriched/selected cells as described in more detail herein. In various embodiments, after having undergone selection the cells may be primed for activation via environmental pressures or antibody co-stimulation. Antibody co-stimulation may be done sequentially or concurrently with isolation using antibody conjugated or physical coated beads, labels, surfaces, and/or particles bound to target cells depending on the various embodiments.

In various embodiments, the cell therapy product (e.g., a heterogeneous mixture of blood components) may be subjected to a Ficoll-based density gradient separation as part of lymphocyte enrichment 104. In various embodiments, the separation process may be performed using a closed, automated cell processing instrument such as the Sepax® 2 system, which operates under sterile conditions using a standard aseptic tubing kit. In various embodiments, a result of the lymphocyte enrichment 104 step may be to isolate and concentrate the mononuclear cell fraction, which includes lymphocytes and monocytes. In various embodiments, the lymphocyte enrichment may include removing unwanted components such as red blood cells (RBCs) and granulocytes.

In various embodiments, the Ficoll separation protocol, often referred to as the NeatCell™ Program, may be designed to yield a highly enriched population of lymphocytes with consistent cell viability and recovery. For example, in various embodiments the NeatCell™ C-Pro protocol software by Cytiva™ may be used with the Sepax™ T-Pro instrument for isolating the mononuclear cells fraction of blood and cellular products via a density gradient medium. The Sepax™ T-Pro instrument may accept an initial bag (e.g., a flexible single-use container for apheresis, bone marrow, umbilical cord, or other starting material) and one or more single-use kits for washing and processing and be used in various embodiments. Other commercially available products may be available to carry out the methods described herein according to various embodiments. During the lymphocyte enrichment 104 process, cells may also be washed to remove residual Ficoll and other process-related impurities according to various embodiments. Such impurities may otherwise interfere with downstream applications such as T cell activation and genetic modification.

In various embodiments, lymphocyte enrichment 104 may use magnetic beds for isolating T cells from a mixed cell population by selectively binding unwanted cells with antibodies and then using magnetic beads to separate them from the desired T cells. In various embodiments, selection may be carried out by positive selection (e.g., isolating T cells directly). In various embodiments, selection may be carried about by negative selection (e.g., depleting non-T cells). In various embodiments, a combination of positive and negative section may be incorporated into the lymphocyte enrichment 104 methods described herein.

In various embodiments, negative selection may use more than one antibody (e.g., an antibody cocktail) used to bind a variety of different cell types where the different cell types do not comprise T cells.

In various embodiments, positive selection may include use of antibodies specific to a T cell surface marker to directly label T cells.

In various embodiments, for negative selection, lymphocyte enrichment 104 process may comprise the step of magnetic separation. There are a variety of commercially available instruments (e.g., KingFisher™, DynaMag™ magnetic racks, etc.) which may be used with commercially available magnet beads (e.g., Dynabeads™).

For example, in the lymphocyte enrichment 104 process, biotinylated antibodies may be used to bind non-T cells according to various embodiments. In various embodiments, streptavidin-coated magnetic beads many then be added to bind to the biotinylated antibodies. In various embodiments, the bound non-T cells may be attracted to a magnetic field while the T cells remain in a supernatant.

In various embodiments, for positive selection, lymphocyte enrichment 104 process may comprise the step of magnetic separation. For example, antibodies specific for T cells (e.g., anti-CD4, anti-CD8, and/or anti-CD3) may be conjugated to magnetic beds may bind to the desired T cells in various embodiments. In various embodiments, the bound T cells may be attracted to a magnetic field and the non-T cells may be washed away.

Use of magnetic bead isolation may require additional steps to remove the added impurities according to various embodiments. For example, instruments exist specifically to implement cell isolation and bead removal (e.g., Gibco™ CTS DynaCellect™ Magnetic Separation System) which may be used in the lymphocyte enrichment 104 process according to various embodiments.

In various embodiments, a T cell selection/enrichment process may include CD4+/CD8+ T cell enrichment carried out via magnetic bead or acoustic selection isolation with a T cell recovery of 30-80% (relative to incoming apheresis T cell composition) at a T cell purity of more than 85% and viability of typically above 90%.

In various embodiments, the enriched lymphocyte population may then then be formulated in a suitable growth medium, preparing the cells for subsequent activation and transduction steps. For example, a formulation may be deposited into an intermediate and/or final single-use bag of the Sepax C-Pro™ instrument or an instrument designed for use with Dynabeads™.

In various embodiments, the lymphocyte enrichment 104 process may be conducted in a closed system within an ISO 7 cleanroom environment, with all fluid connections made using sterile tubing welders, aseptic connections, or performed in an ISO 5 laminar flow hood. Other systems may also be suitable for ensuring a high level of sterility that minimizes the risk of environmental contamination.

c. Activation

In various embodiments, the T cell manufacturing process 100 may include a T cell activation 106 step. In various embodiments, T cell activation 106 may be used in an autologous or an allogenic T cell manufacturing process 100. In various embodiments, T cell activation 106 may prepare the lymphocytes for efficient genetic modification and subsequent expansion. In various embodiments, T cell activation 106 may begin after lymphocyte enrichment 104. In various embodiments, T cell activation 106 may use an enriched lymphocyte population as a starting material. In various embodiments, T cell activation 106 step of the T cell manufacturing process 100 may be conducting in a closed, sterile single-use system such as one of those described herein.

In various embodiments T cell activation 106 may be initiated by exposing the T cells to a combination of stimulatory agents. In various embodiments, T cell activation 106 may be completed in a serum-free medium. In various embodiments, one or more stimulatory agents may be used for T cell activation 106. In various embodiments, a stimulatory agent may include an anti-CD3 monoclonal antibody. In various embodiments, a stimulatory agent may include interleukin-2 (IL-2). In various embodiments, IL-2 may include a recombinant human IL-2.

In the T cell activation 106 step, the anti-CD3 antibody may bind to a CD3 complex on the surface of T cells according to various embodiments. Binding may operate by mimicking antigen recognition and triggering a T cell receptor signaling cascade. In various embodiments, the signal may be required for initiating T cell activation. In various embodiments, the signal may also increase cell proliferation and/or survival.

IL-2 may be used as a T cell growth factor that supports the expansion and sustains the activated state of the T cells according to various embodiments. In various embodiments, a cell culture medium may include OpTmizer™ or AIM-V® and then may be supplemented with defined additives to support cell viability and function without the variability introduced by human serum.

In various embodiments, T cell activation 106 may include other environmentally controlled factors. For example, T cell activation 106 may be carried out at or near physiological conditions. In various embodiments, T cell activation 106 may be carried out at a temperature at or around 37° C. In various embodiments, T cell activation may be carried out at about 5% CO₂. In various embodiments, the T cell activation 106 step may occur over approximately 48 hours. In various embodiments, a T cell activation step may be facilitated by one or more of the systems described herein or elsewhere. In various embodiments, a system may include a humidified incubator.

In various embodiments the T cell activation step 106 may result in the T cells undergoing a phenotypic change, including upregulation of activation markers (e.g., CD25, CD69) and increased metabolic activity. In various embodiments, such changes render the T cells more receptive to transduction (e.g., retroviral, lentiviral transduction, etc.). In various embodiments, the T cell activation 106 step may also help reduce or eliminate non-T cell populations.

In various embodiments, the T cell activation step 106 may increase the success of the entire T cell manufacturing process 100. For example, T cell activation 106 may influence transduction efficiency, expansion kinetics, and the functional quality of the final cell therapy product.

d. Transduction

In various embodiments, the T cell manufacturing process 100 may include a transduction 108 step. As cited herein, various publicly available descriptions of systems and methods may be incorporated into the workflow detailed herein such as those in US20150344844A1 and US20170136063A1. In various embodiments, transduction may comprise one or more of either or both of transduction and transfection steps. In various embodiments, activated T cells may be genetically modified to express a therapeutic receptor. In various embodiments, the therapeutic receptor may include a chimeric antigen receptor (CAR) or a T cell receptor (TCR). In various embodiments, the transduction 108 step may be used to introduce a gene encoding a receptor. In various embodiments, the receptor may enable the T cells to recognize and attack specific target cells, such as cancer cells.

In various embodiments, the process may begin with the preparation of a sterile, closed-system culture bag, which may pre-coated with a recombinant human fibronectin fragment, such as RetroNectin® when using retroviral vectors. The coating may facilitate the co-localization of the viral vector and the T cells, thereby, enhancing the efficiency of gene transfer in various embodiments. In various embodiments, the fibronectin fragment may be applied at a concentration of 10 μg/mL and incubated at 2-8° C. for 20±4 hours. After coating, the bag may be washed to remove excess fibronectin and then incubated with the viral vector according to various embodiments. Examples of retroviral vectors commonly used may include but are not limited to a gamma-retrovirus like MSGV-FMC63-28Z or MSGV-FMC63-CD828BBZ. In various embodiments, incubation may occur for approximately 3 hours at 37° C. and at 5% CO₂.

Following vector incubation, the activated T cells may be introduced into the same bag without removing the viral supernatant. The transduction 108 step may be carried out in a serum-free medium (e.g., OpTmizer™) in various embodiments. In various embodiments, environmental conditions may be maintained at or around 37° C. and/or 5% CO₂. In various embodiments, a single transduction cycle may last approximately 20 hours. In various embodiments, a multiple transduction cycle approach may be used in the T cell manufacturing process 100.

In various embodiments, a different method of co-localization may be employed. For example, a pinning a viral vector molecule and a target cell molecule on a membrane (e.g., see U.S. Patent Publication No. US20230080444A1, which is hereby incorporated by reference in its entirety and for all purposes) according to various embodiments. Such a pinning method may be employed for lentiviral vectors and other vectors.

The method in US20230080444A1 describes a specialized device that may use a semi-permeable membrane to spatially co-localize cells and viral particles, thereby enhancing the probability of successful gene transfer. The specialized device described in US20230080444A1 may be incorporated into the workflows described herein according to various embodiments. In various embodiments, a transduction 108 step may begin by introducing a fluid comprising both the target cells and lentiviral particles into a flow chamber adjacent to the membrane. In various embodiments, the membrane may be engineered with pores small enough (about 550 nm) to prevent the passage of both cells and viral particles, ensuring they remain concentrated on its surface.

In various embodiments, the transduction 108 step may use convective flow to direct the cell-virus mixture across the membrane surface to form a monolayer of cells and viral particles. The co-localization may increase the likelihood of viral entry into the cells before the virus degrades according to various embodiments. The flow rate within the specialized system may be between about 15-25 μL/min/cm² for lentiviral vectors (which are ˜100 nm in diameter) to optimize the balance between nutrient replenishment and viral concentration at the membrane interface according to various embodiments.

After a defined incubation period (e.g., 90 minutes), a recovery fluid may be introduced from the opposite side of the membrane to lift and suspend the transduced cells according to various embodiments. The fluid flow may be applied in both a normal and transverse direction to maximize recovery efficiency according to various embodiments. The recovered fluid including the transduced cells and residual viral particles may be processed further to separate the cells from unbound virus according to various embodiments. Separation may be facilitated either through centrifugation or a secondary membrane-based filtration device according to various embodiments.

In various embodiments, the transduction 108 step may be modified for use with lentiviral vectors and include a transfection and/or a transduction step. For example, in various embodiments, an electroporation and/or a transduction step may include transfecting concentrated cells (e.g., 15-300 e6/mL) with genetic or non-genetic material (e.g. DNA or RNA encoding ZFN or CRISPR or TALENs) to affect the desired gene modifications (e.g., gene knockout and/or additions). Post-electroporation, the cells may be washed, buffer exchanged, and/or diluted to minimize exposure to the electroporation buffer during transduction. In various embodiments, the T cells may be transduced with construct-encoding lentiviral vectors or retroviral vectors using enhancing reagents (e.g., retronectin, protamine sulfate, polybrene, or vectofusin-1, etc.) at optimized conditions or enhancer-free physical co-localization viral vector-based gene delivery methods at a cell to vector ratio designed to achieve desired transduction efficiencies and genomic integration. The volume of viral vector may be controlled at a target multiplicity of infection (i.e. transducing viral particle units per cell) and incorporated into the transduction system or the culture system. The transduction seed density may typically be between 1-5 e6 cells/mL to achieve the desired particle per cell unit ratio and may last from 1 hour to 72 hours at temperature ranges from 15° C. to 37° C.

After the transduction 108 step in the T cell manufacturing process 100, the cells may be washed to remove residual vector particles and/or process-related impurities according to various embodiments. The transduced T cells may then be transferred to a fresh culture bag for expansion 110. In various embodiments, the entire transduction process may be performed in a closed system using serum-free media. The closed systems described herein and elsewhere may enhance good manufacturing practice (GMP) compliance, scalability, and safety for clinical applications.

e. Expansion

In various embodiments, the T cell manufacturing process 100 may include an expansion 110 step. In various embodiments, expansion 110 may be subsequence to transduction 108. In various embodiments, expansion 110 and transduction 108 overlap. In various embodiments, transduction 108 and expansion 110 may occur concurrently. In various embodiments, the step of expansion 110 may be completed on suitable equipment that may be commercially available such as in shake flasks, rocking wave bioreactors, or stirred tank bioreactors. Systems may be selected based on the application (e.g., stirred tank bioreactors may allow for increased volume) in accordance with various embodiments. In various embodiments, the transduced cells may be pre-washed or directly enter an expansion step utilizing various batch, batch-fed, perfusion, and solera methods to obtain a sufficient number of cells to meet a dose specification.

An expansion 110 step may be used in autologous and/or allogeneic T cell manufacturing 100 processes according to various embodiments. Expansion 110 may be used to increase the number of transduced T cells to a therapeutically effective dose while maintaining their viability, functionality, and desired phenotypic characteristics in accordance with various embodiments.

The expansion 110 step may be completed in any closed system described herein or elsewhere. In various embodiments, a close system may comprise one or more single-use cell culture bags. In various embodiments, the expansion 110 step may a serum-free culture medium to minimize contamination risks and variability associated with serum-based media. In various embodiments, expansion 110 may comprise the step of incubating the cell therapy product at a temperature of approximately 37° C. In various embodiments, expansion 110 may comprise the step of incubating the cell therapy product at a CO₂ concentration of 5%.

In various embodiments, expansion 110 may be carried out over a three-day period. In various embodiments, expansion 110 may be carried out over a six-day period. In various embodiments, expansion 110 may be carried out between three to six days. In various embodiments, expansion 110 may be carried out in less than three days. In various embodiments, expansion 110 may be carried out in more than a six-days. The duration of the expansion 110 step may depend on the dose, cell type being used, or any other parameter known to impact expansion 110 times.

In various embodiments, the expansion 110 step may be carried out in the presence of IL-2. In various embodiments, expansion may be carried out in the presence of other support factors (e.g., IL-15)

In various embodiments, the expansion 110 step may lead to generation of a cell therapy product suitable for formulation and/or cryo preservation allowing for storage and/or transport of the cell therapy product to location (e.g., a hospital or clinic) suitable for administration of the cell therapy product to a patient.

f. Final Formulation

In various embodiments, the T cell manufacturing process 100 may include a final formulation step 112. In various embodiments, a final formulation may comprise a cell therapy product suitable for administration to a patient. In various embodiments, a final formulation step may occur after a sufficient number of target engineered cells are achieved to meet a dose specification.

In various embodiments, a final formulation 112 may include an additional depletion step. In various embodiments, the depletion step may help adjust a final formulation concentration to therapeutic concentrations. In various embodiments, the expanded cells may be washed via centrifugation, buffer exchange, or acoustic separation to achieve a desired cell concentration. In various embodiments, a concentration of 50-300 e9 cells/mL in 200-500 mL of media or other concentrations may be desirable.

In various embodiments, formulation may involve a harvest wash step. In various embodiments, a commercially available system be incorporated into the T cell manufacturing process 100. Examples of commercially available systems include but are not limited centrifugation resuspension using Cytiva Sepax™ Culture Wash or Sefia FlexCell™, perfusion dilution using Cytiva Xuri WAVE™ system or Applikon Biosep™ cell retention, or buffer exchange inertial flow fluid dynamics. After the wash step, a dose specific post-wash volume may be combined with cryo protectant reagents and buffers at a specified ratio in a closed vessel according to various embodiments. For final formulation 112, the step may occur in using the Terumo FINIA™ system, a buffer exchange inertial flow device, or traditional manual methods using gravitational, pump, or syringe fluid handling techniques. The final product bags may be cryo preserved for storage, shipment, and/or later use.

g. Exemplary Method

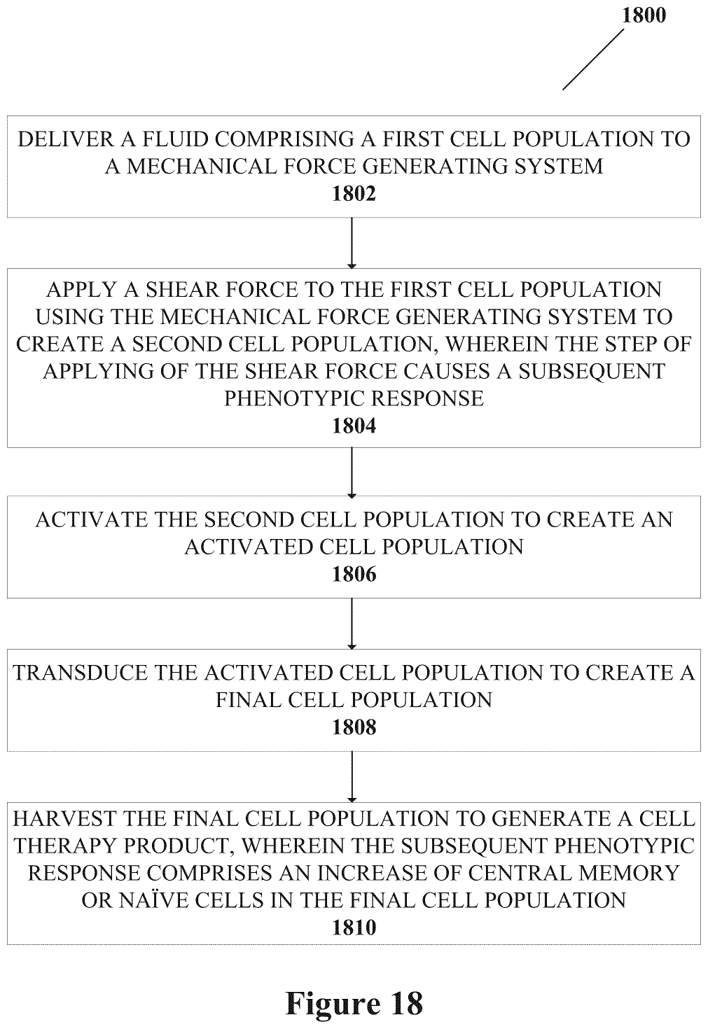

FIG. 18 illustrates a method of applying a shear force to a first cell population to cause a subsequent phenotypic response according to various embodiments.

Step 1802 of the method comprises delivering a fluid comprising a first cell population to a mechanical force generating system according to various embodiments.

Step 1804 of the method comprises applying a shear force to the first cell population using the mechanical force generating system to create a second cell population, wherein the step of applying the shear force causes a subsequent phenotypic response according to various embodiments.

Step 1806 comprises activating the second cell population to create an activated cell population according to various embodiments.

Step 1808 comprises transducing the activated cell population to create a final cell population according to various embodiments.

Step 1810 comprises harvesting the final cell population to generate a cell therapy product, wherein the subsequent phenotypic response comprises an increase of central memory or naïve cells in the final cell population according to various embodiments.

In various embodiments, the increase of central memory cells is 0.3% to 16.3% and the increase of naïve cells is 0.3% to 2.5%.

In various embodiments, the subsequent phenotypic response comprises a decrease of effector cells in the final cell population. In various embodiments, the decrease of effector cells is 0.2% to 12.9%.

In various embodiments, the subsequent phenotypic response comprises an increase of T cells and a decrease of both B cells and NK cells in the final cell population. In various embodiments, the increase of T cells is 2.1% to 10.2%, the decrease of B cells is 0.3% to 4.5%, and the decrease of NK cells is 0.3% to 2.0%.

In various embodiments, the subsequent phenotypic response comprises an increase of CD69 or CD25 expression in the activated cell population. In various embodiments, the increase of CD69 is 2% to 4% and the increase of CD25 is 5% to 14%.

In various embodiments, the subsequent phenotypic response comprises an increase of PD-1 expression. In various embodiments, the increase of PD-1 expression is 0.6% to 7.1%.

In various embodiments, the subsequent phenotypic response comprises an increase of CD27 or CD28 expression. In various embodiments, the increase of CD28 expression is 0.3% to 3.4%.

In various embodiments, the step of transducing comprises using a lentiviral vector or a retroviral vector.

In various embodiments, the first cell population comprises T cells.

In various embodiments, the first cell population comprises peripheral blood mononuclear cells (PBMCs).

In various embodiments, the step of applying the shear force occurs for 30 minutes to 120 minutes. In various embodiments, the step of applying the shear force occurs for 60 minutes to 120 minutes.

In various embodiments, the step of applying the shear force comprises applying at a shear rate of 569.78 s⁻¹ to 10,533.53 s⁻¹.

In various embodiments, the step of applying the shear force comprising applying at a shear rate of 614.75 s⁻¹.

In various embodiments, the step of applying the shear force comprises applying a total shear exposure of 1,106,557 s to 4,426,230 s.

In various embodiments, the mechanical force generating system comprises a housing for storing the cell population.

In various embodiments, the mechanical force generating system comprises a fluid channel having a first end and a second end, wherein the first and second ends are fluidically connected to the housing.

In various embodiments, the mechanical force generating system comprises a pump positioned along the fluid channel or in the housing.

In various embodiments, the inner diameter of the fluid channel is 4.8 mm to 25.4 mm.

In various embodiments, the length of the fluid channel is 45 cm.

In various embodiments, the pump operates to produce a fluid flow rate of 400 mL/min to 55,000 mL/min.

In various embodiments, the pump comprises a peristaltic pump.

In various embodiments, the method may generate an improved cell therapy product made by delivering a fluid comprising a first cell population to a mechanical force generating system, applying a shear force to the first cell population using the mechanical force generating system to create a second cell population, wherein the step of applying of the shear force causes a subsequent phenotypic response, activating the second cell population to create an activated cell population, transducing the activated cell population to create a final cell population, and harvesting a final cell population to generate a cell therapy product, wherein the subsequent phenotypic response comprises an increase of central memory or naïve cells in the final cell population.

In various embodiments, the method may generate an improved cell therapy product, wherein the improved cell therapy product comprises a population of shear force treated cells having an increase of CD69 expression, CD25 expression, PD-1 expression. CD27 expression, CD28 expression, IL-2 expression, CD4 expression, CD8 expression, naïve T-cells, central memory T cells, central memory T cells, or a combination thereof.

In various embodiments, a method of applying a shear force to a first cell population to cause a subsequent phenotypic response, the method comprising delivering a fluid comprising a first cell population to a mechanical force generating system, step for applying a shear force to the first cell population using the mechanical force generating system to create a second cell population, wherein the step for applying the shear force causes a subsequent phenotypic response, activating the second cell population to create an activated cell population, transducing the activated cell population to create a final cell population, and harvesting a final cell population to generate a cell therapy product, wherein the subsequent phenotypic response comprises an increase of central memory or naïve cells in the final cell population.

### IV. Examples of Systems and Methods for Application of Mechanical Forces During a Cell Therapy Manufacturing Process

In various embodiments, the systems and methods described herein may be directed to application of mechanical forces (e.g., shear forces) to a cell therapy product. Application of mechanical forces have been generally considered a bad idea in the industry because it can lead to cell damage and even cell death. However, when shear forces are applied in a specific way (e.g., for specific time periods, at specified quantities, and for a total accumulated force quantity) there may be positive benefits according to various embodiments. Described herein are examples of systems and methods used to generate the total accumulated shear force in a way that positively alters a phenotypic population of cells to improve a cell therapy product.

In various embodiments, peristaltic pumping systems and methods were used to perform a variety of tasks in a cell therapy manufacturing process. For example, peristaltic pumps were used to transfer fluid between single-use bags through tubing (i.e. consumable kits) to facilitate a T cell manufacturing process. In various embodiments, precise fluid transfers were used for reduction or elimination of exposure to the environment. For example, a bench top peristaltic pump (MasterFlex® L/S) was used in conjunction with the various embodiments described herein. As described herein, a peristaltic pump was used to subject cell cultures to continuous exposure to fluidic shear stress leading to a total accumulated shear force. In various cases, a peristaltic was used to subject cell cultures to intermittent exposure to fluidic shear stress. T cells (n=3) and PBMCs (n=3) in the fresh (n=3) and cryo preserved (n=3) format were used as starting material for the examples herein. All materials were sourced from healthy donors. The starting material was activated for a minimum of 48 hours using a CD3 and a CD28. The cell cultures were exposed to peristaltic pumping at 400 mL/min at three experimental durations: normal (30 mins), high (60 mins), and extreme (120 mins). The control conditions included not exposing the cells to any pumping (i.e. shear force). Cells were expanded in cell culture bags in static incubators for six additional days. Cell counts and analytical samples were taken at designated points. T cell activation, phenotype, CD4:CD8 ratios, PD-1 expression, CD27/CD28 costimulatory markers, and IL-2 in suspension were analyzed. Regardless of starting material and condition, cell cultures exposed to the extreme duration (120 mins) saw an increase in cell viability, growth, activation, phenotype, IL-2 production, and other supporting cell surface markers.

a. Mechanical Force Generation

In various embodiments, mechanical stress (e.g., shear stress or shear force) may be applied to cell using a variety of means (e.g., shaking, rocking, stirring, etc.). In these examples, we focus our attention to mechanical stress applied using one or more peristaltic pumps. However, a skilled artisan will appreciate that a variety of different pumps or motors may be used to drive application of mechanical stress. It is noteworthy that the pump system described herein required customization due to lack of available commercially existing solutions (e.g., systems capable of applying higher than typical amounts of shear force). Examples of other pump systems may include, but are not limited to, gear drives, diaphragm pumps, centrifugal pumps, submersible pumps, piston pumps, screw pumps, lobe pumps, displacement pumps, reciprocating pumps, rotary driven pumps, drum pumps, turbine pumps, etc. During peristaltic pumping, there are several forces that contribute to shear stress by nature of a positive displacement pump according to various embodiments. For this reason, we selected the mechanical force generating systems described herein to carry out the various methods described in the examples herein.

In various embodiments, compression force may a primary force in peristaltic pumping. In various embodiments, force may be generated through properties of the mechanical force generating system overall. In various embodiments, the properties may include tubing parameters such as size, length, and geometry. In various embodiments, the properties may include size and shape of the flexible housing. A skilled artisan in the field will appreciate that numerous ways exist to generate mechanical forces through equipment selection and modification. For example, the force exerted on a fluid within the flexible tube by the rollers or shoes may be adjusted easily through rate changes, geometry changes, and/or component size selection according to various embodiments. For example, the force may be generated by squeezing the tube which pushes the fluid forward according to various embodiments. For an additional example, the strength of forward force may depend on factors such as the size and material of the tubing, and the squeezing pressure by the rollers or shoes according to various embodiments.

In various embodiments, frictional force may occur as the rollers or shoes move along the tubing.

In various embodiments, inertia may be another force that may be generated in high-speed peristaltic pumping applications. As the rollers or shoes accelerate or decelerate, they may impart inertia forces on the fluid and tubing, which can affect flow dynamics in accordance with various embodiments.

In various embodiments, viscous resistance may occur if the fluid has high viscosity, and it may exert a resistance to flow when being pumped. The diameter and length of the tubing and the flow rate can contribute to viscous resistance according to various embodiments. The various embodiments described herein may overcome this force to help maintain a steady flow rate. Elasticity of the tubing material may contribute to the pumping process by allowing the tube to rebound after compression which may help draw fluid into the pump and maintain constant flow according to various embodiments.

b. Cell Culture Parameters

Healthy donor, fresh leukopak without age, gender, ethnicity, BMI restrictions were purchased (AllCells™, CA, USA). T cell isolation was performed using the Sepax™ C-Pro Cell Processing Instrument (Cytiva™, MA, USA) and the CliniMACS™ Plus System (Miltenyi Biotec™, CA, USA). Positive CD3+ selection was performed according to Miltenyi's instructions. PBMC selection used the Sepax™ C-Pro Cell Processing Instrument (Cytiva™, MA, USA) and Lymphocyte Separation Medium (Lonza™, CH) to perform a density gradient separation. OriGen™ Biomedical PermaLife™ cell culture bags (OriGen™, TX, USA) were used for cell culture. OriGen™ PL325 bag was used for activation and the PL120 size bags were used to expand each experimental condition after exposure to shear stress. All cell culture bags were placed in an incubator at 37.0° C. and 5.0% CO2. The cell culture was grown in Gibco™ CTS OpTmizer™ cell expansion media supplemented with Intraleukin-2. Samples were withdrawn from the cell culture bags at pre-determined time points throughout the culture period for cell counts. A portion of the sample was centrifuged, resuspend in CryoStor™ CS10 (Biolife Solutions™), and stored at −80° C. for future analytical testing. The NucleoCounter™ NC-200 (ChemoMetec™ A/S, Denmark) and the Vial-Cassette™ containing acridine orange (AO) and 4′,6-diamidino-2-phenylindole (DAPI) were used to measure the cell density, viability, diameter, and aggregates.

Multiple types of tubing materials were tested with the Masterflex® L/S pump and the pump head, including, C-Flex, silicone, Tygon®, Viton™, neoprene and PharMed®. Fluid compatibility, fluid temperature, system pressure, motor rpm, clarity of tubing, duty cycle, maintenance, tubing purity, and viscosity were all factors considered when selecting a tubing. The factors that were used to determine the correct type of tubing to use in the experiments described herein were based on the available material that is commonly used for cell culture. For example, silicone tubing was selected due to its excellent biocompatibility, lack of leachable additives, dioctyl phthalate, and other plasticizers. Silicone tubing is odorless, nontoxic, and does not impart taste to the exposed fluids. Silicone tubing has a broad static temperature working range (e.g., −50° C. to 230° C.) and a broad pumping working range (e.g., −40° C. to 100° C.). Silicone tubing also includes an ideal or close to ideal gas permeability for the purposes described herein and its translucent nature made it a good choice for the experiments because it allows visual inspection. Gas can permeate into the tubing while the cells were exposed to peristaltic pumping and the cell culture was visually observed in the tubing.

c. T Cell Activation

Both T cells and PBMCs were activated. There may be multiple methods to activate these cells, including, the methods detailed herein and elsewhere. Based on the ease of procuring Miltenyi™ products, we used soluble anti-CD3 and anti-CD28. Research has shown that anti-CD3 and anti-CD28 may be needed for complete T cell activation. Research has shown that complete PBMC activation may occur by solely using anti-CD3. Further, as described herein, there may be some methods allowing for transduction without an activation step.

TABLE 1
Activation Reagent Testing
Activation reagent

| | Duration | | | | | |
|---|---|---|---|---|---|---|
| | 24 | 24 | 48 | 48 | 72 | 72 |
| | hours | hours | hours | hours | hours | hours |
| Quantity of | 100 | 250 | 100 | 250 | 100 | 250 |
| anti-CD3 | mg | mg | mg | mg | mg | mg |
| Quantity of | 250 | 250 | 250 mg | 250 | 250 | 250 |
| anti-CD28 | mg | mg | | mg | mg | mg |

Table 1 details the activation conditions tested to optimize activation reagent quantities and time durations. After analyzing the results, 48 hours of activation with 250 mg of both anti-CD3 and anti-CD28 were used in all experiments.

The T cell activation step was completed between 24 to 48 hours to fully activate the cells. In addition to the incubation duration, the concentration of anti-CD3 and anti-CD28 were varied to determine the optimal amount of reagent to use as described in Table 1. Careful consideration given to ensure that the T cells were not over activated by monitoring T cell activation surface markers such as PD-1, CD69+, and CD25+. This screening determined that at least 48 hours of activation incubation with 250 mg of anti-CD3 and 250 mg of anti-CD28 resulted in an acceptable level of activation.

d. Mechanical Force Generating Systems

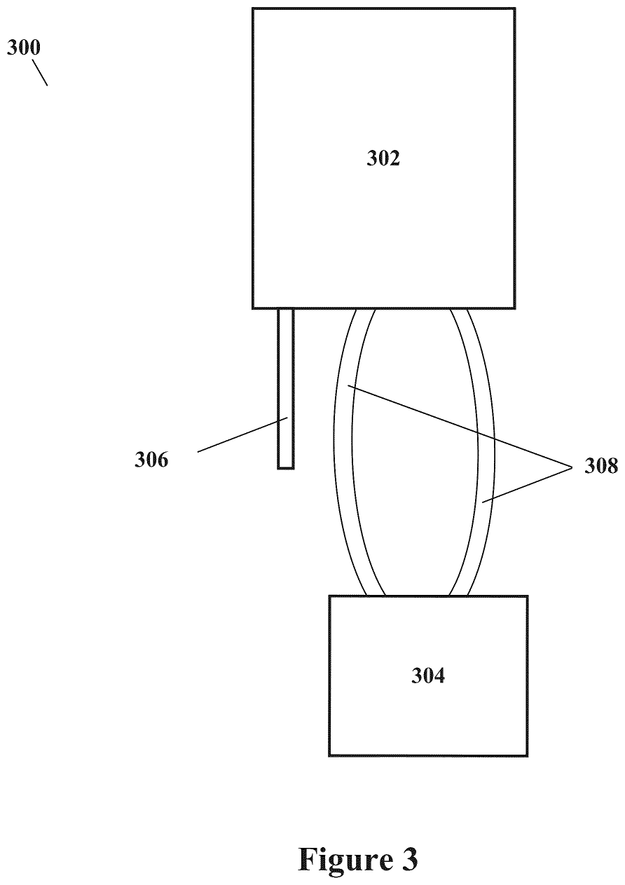

FIG. 3 illustrates a mechanical force generating system 300 including a flexible housing 302, a pump 304, a sampling port 306 and fluid channel 308 according to various embodiments. In various embodiments, the mechanical force generating system 300 may operate by circulating a fluid from the flexible housing 302 through the fluid channel 308 using a pump 304 to generate mechanical force to act upon the fluid. In various embodiments, a sampling port 306 may allow for fluid sampling at various timepoints.

In various embodiments, a mechanical force generating system 300 may include a commercially available consumable kit designed to work with any of the systems described herein (e.g., a Sepax™ C-Pro processing instrument manufactured by Cytiva™). In various embodiments, the flexible housing 302 may include a single-use bag designed to hold a cell therapy product, apheresis, or other some other starting material (e.g., leukopak). In various embodiments, the pump 304 may include a peristaltic pump (e.g., a Watson-Marlow™ 120U Peristaltic Pump or a Masterflex® L/S Peristaltic Pump from Cole-Palmer®). In various embodiments, a fluid channel 308 may include any of the different kinds of flexible tubing described herein and elsewhere. In various embodiments, a sampling port 306 may allow aseptic fluidic connectivity to any other systems in a cell therapy workflow. In various embodiments, additional components may be aseptically connected to the mechanical force generating system 300. In various embodiments, the mechanical force generating system 300 may be one component in a large set of system components in a cell therapy manufacturing system (e.g., connecting either or both manual and automated systems for manufacturing cell therapy products). In various embodiments, any of the components of the mechanical force generating system 300 may be fluidically connected through aseptic connectors, welds, etc.

For the examples described herein, a peristaltic pump and tubing system for generating shear stress was used according to various embodiments. The system may expose cell cultures to continuous peristaltic pumping by recirculating a fluid comprising cells though the fluid channel. For the examples described herein, a cell culture bag 1000 mL was used as the flexible housing. For the examples described herein, the sample port allowed for cell culture to be removed from the mechanical force generating system. For the examples, the fluid channel included a recirculation loop from MasterFlex® and was size 15 tubing at 45 cm in length.

We developed this to continuously expose the cells to peristaltic pumping without any additional interference so that we could both qualitatively observe (e.g. through visual inspection) and quantitatively observe (e.g., through marker quantification) phenotypic properties. This system design allowed samples to be taken during operation through the sample port. These samples were used for cell counts, analytical processing, and to seed the expansion cell culture. A 1000 mL flexible housing (i.e. a bag) 1000 mL bag was selected from Charter Medical™ to include the initial culture volume. The peristaltic pump moved the cell culture in a counterclockwise direction, but a skilled artisan in the field will appreciate that any direction may be select, including, clockwise, counterclockwise, or a combination of the two.

e. Pump Selection

In various embodiments, there may be many variables involved in selecting peristaltic pump tubing to optimize tubing lifespan and fluid transfer performance. The correct tubing material may be selected for the specific application according to various embodiments. The type of fluid that the tubing is exposed to, expected pump duration, and system pressure may need to match the tube's attributes according to various embodiments. In various embodiments, a system pressure of both the inlet and outlet may be considered and monitored in order to prevent over pressurization or excessive vacuum which could damage the tube.

A variety of different pumps may be available for use in cell therapy applications, however, peristaltic pumps have maintained industry dominance. Peristaltic pumps were used in the examples provided due to their compatibility with tubing (e.g., ability to move fluids through flexible tubing by applying rollers against the flexible tubing), precise reproducible dosing, ease of maintaining sterility within a closed system, consistent flow rates, and operational compatibility with different tube types. Although peristaltic pumps may be known in the industry for reducing mechanical forces (e.g., shear forces), our examples include methods of increasing application of shear forces by sometimes operating peristaltic pumps outside of typically operational parameters (e.g., pump speeds and durations).

The Watson-Marlow™ 120U Peristaltic Pump (Wilmington, MA, USA) and Masterflex® L/S Peristaltic Pump from Cole-Palmer® were selected to be used in the experiments described herein. The L/S Peristaltic Pump used the Easy-Load® II pump head (model 77200-62) and Masterflex® L/S-15 silicone tubing (cat no. 94610-15).

Watson-Marlow™ 120U Peristaltic Pump (Wilmington, MA, USA) was selected initially, but was not able to perform as needed for our experiments without modification. This pump may be commonly used in the pharmaceutical industry and may often be incorporated in cell therapy systems. The maximum flowrate range of a Watson-Marlow™ 120U Peristaltic Pump is 180 ml/min and was determined to not fit within the experimental range of 100 mL/min to 400 mL/min.

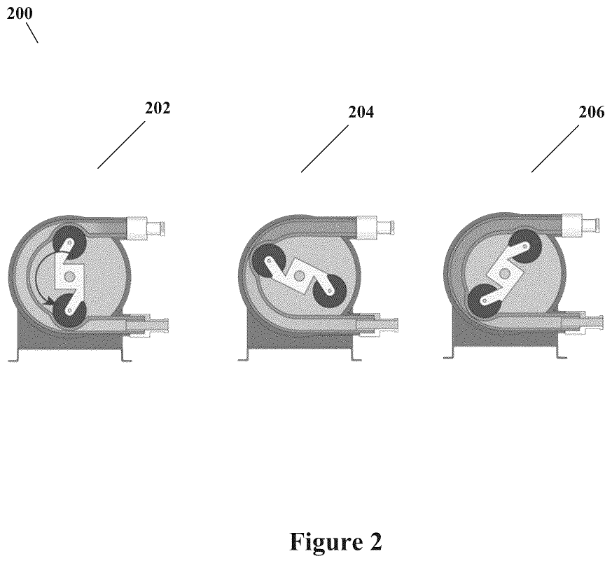

FIG. 2 illustrates a peristaltic pump 200 in three operational positions 202, 204, 206 according to various embodiments. As shown in FIG. 2, flexible tubing may be mounted to a peristaltic pump in such a way that rollers of the peristaltic pump interact with the flexible tubing by compressing it and moving the rollers to drive fluid through the tubing. In a first position 202 the rollers may be in a starting position and move to a second positive 204 and finally to a third position 206 to draw fluid through the flexible tubing. As described herein, there are many advantages to using a peristaltic pump 200 in combination with flexible tubing which include, but is not limited to, precise flow rates, in some cases reduces mechanical forces on cells, and ability to maintain sterility in a closed system.

In a peristaltic pump, fluid is transported by compressing a flexible tube with rollers or shoes according to various embodiments. Peristaltic pumps are also known as a roller pump which uses compression to generate moving regions of high pressure that pushes the fluid through the tube according to various embodiments. In various embodiments, peristaltic pumps use a tube, the contents of the tube may not come into contact with the pump components which protects exposure of the fluid in the tube.

In various embodiments, peristaltic pumps transport fluid by continuously compressing a tube with a set of rollers or shoes. In various embodiments, this motion may create a vacuum through rotary motion and draws the fluid into the tube/pump. In various embodiments, the fluid may enter the tube, a roller follows immediately and pushes the fluid to the outlet of the tube. In various embodiments, the next roller squeezes an empty portion of the tube, creating a vacuum and repeating the entire process. In various embodiments, rollers may be evenly distributed along a 360-degree arrangement whereas more rollers can reduce pulsation of the fluid. An additional benefit of the peristaltic pump design may be that, even without a check valve, they are immune to backflow when not pumping. In various embodiments, advantages of peristaltic pumps may be resistant to leaks, capable of running dry for extended periods, handle very viscous fluids, may not allow contamination, may be resistant to backflow, and may be low cost to maintain.

In various embodiments, peristaltic pump rollers may be fixed occlusion. In various embodiments, peristaltic pump rollers may be spring-loaded rollers. Fixed occlusion rollers do may change position as the pump rotates such as the pumps. This may cause the pressure they place on the tubing to be constant according to various embodiments. Occlusions can ultimately shorten the lifespan of the tube therefore selection of the correct tube thickness is critical. Spring-loaded rollers may include hinged rollers and tension may be placed on them via a spring according to various embodiments. These rollers may exert a force on the tubing that correlates to the tubing thickness according to various embodiments. Such a configuration may allow for constant pressure regardless of thickness and size, preventing premature tube wear.

f. Tube Selection

In various embodiments, commercially equipment may be used to generate the mechanical forces described in these examples, however, heavy modification may be required since the flow rates and shear forces far exceed those used in standard cell therapy manufacturing methods. In various embodiments, commercially available equipment and technologies may use known rotary peristaltic pumps with some modification to operational parameters. For example, the Clinimacs Plus™ manufactured by Miltenyi™ may be used according to various embodiments. In various embodiments, a MasterFlex® pump head manufactured by Cole Parmer™ may be used. For example, the Cytiva™ Sefia™ Select Cell Processing System™ may include a MasterFlex® pump such as the versions described in these examples.

In various embodiments, a WatsonMarlow™ pump may be used such as the ones described in these examples.

In various embodiments, a pump drive may be digitally controlled and may allow for high precision and accurate fluid transfers. In the examples described herein, pump drive control and modification to operational input parameters was needed to generate the mechanical forces required for a phenotypic response.

The MasterFlex® L/S Easy-Load® II pump head (model 77200-62) was tested in conjunction with tube sizes 15, 24, 35, 36. The MasterFlex® L/S Easy-Load® II pump head (model 77200-62) has four stainless steel rollers with a working range of 0.02 to 600 rpm and may be used in the various embodiments described herein. The maximum flow rate of 2300 mL/min may be capable with the large tubing size according to various embodiments.

TABLE 2
Tubing Size Selection

| Tubing | Tubing | Flow rate | Flow rate | Maximum |
|---|---|---|---|---|
| name | ID | @ 1 rpm | @ 600 rpm | pressure |
| 15 | 4.8 mm | 1.7 | 1000 | 2.7 bar |
| | ( 3/16″) | | | (40 psi) |
| 24 | 6.4 mm | 2.8 | 1700 | N/A |
| | (¼″) | | | |
| 35 | 7.9 mm | 3.8 | 2300 | 2.4 bar |
| | (⅜″) | | | (35 psi) |
| 36 | 9.7 mm | 4.8 | 2900 | 1.4 bar |
| | (⅜″) | | | (20 psi) |

Tube size selection was based the data seen in Table 2. For the example data, a MasterFlex® L/S Easy-Load® II pump was selected to be used in conjunction with size 15 tubing. The size 15 tubing was manufactured by Cole Parmer™ and included a Masterflex® L/S silicone tubing (cat. No. 94610-15). Here, the fluid flow rate within the tubing generated was operated at a flow rate of 1.7 to 1000 mL/min at 600 rpms. In various embodiments, a tubing I.D. of 4.8 mm allowed for easy integration with other system components of the mechanical force generator.

g. Sampling and Marker Assessment Strategy

In various embodiments, cells such as lymphocytes may express markers that can be detected on the surface cell surface using fluorescence labeled monoclonal antibodies and multicolor flow cytometer. Activation, co-stimulatory molecules, T cell phenotype, and proliferation markers may be common T cell surface markers in various embodiments. To better understand how mechanical forces effect cell phenotype we measured a variety of marker quantity changes which directly translate to cell phenotype. For example, when assessing level of T cell activation we quantified PD-1+, CD69+, and CD25+ expression level change before, during, and after application of mechanical force application.

Some cell surface markers such as those for assessing activation may be transient markers in accordance with various embodiments. Expression of these cell surface markers may be present for a short period of time at specific timepoints. Sampling strategy may be important in capturing that these transient markers which may no longer be present in their full form and under or over represent a sample according to various embodiments. As such, we defined the amount of shear force applied in Table 4 and the sampling strategy in Table 3 for accuracy and consistency. In various embodiments, transient markers may be sampled throughout a cell therapy manufacturing process. Cell counts were performed immediately after sample collection and samples for flow cytometry are cryo preserved for later analysis.

TABLE 3
Sampling Strategy

| | | | | | |
|---|---|---|---|---|---|
| Sample | Day 0 | Day 2 | Day 2 | Day 3 | Day 8 |
| Point | | Post- | Post- | | Harvest |
| (name) | | activation | pumping | | |
| Approx | 0 hours | 48 hours | ~2 hours | 72 hours | |
| Time | | | after post- | | |
| (hours) | | | activation | | |
| Purpose | Baseline | Cell count | Cell count | 24 hours | Harvest, |
| | cell count | to determine | post- | post | end of |
| | to determine | baseline total | exposure | exposure | expansion |
| | total cell | cell density, | to shear | to shear | and run |
| | density, cell | cell viability, | stress. | stress | |
| | viability, and | and cell | This is | | |
| | cell diameter | diameter | the cell | | |
| | | | expansion | | |
| | | | seeding | | |
| | | | value. | | |

In various embodiments, shear for may be applied for a duration of 0, 30, 60, or 120 minutes. In the examples, extreme flow rates of 400 mL/min were be used for all experiments after initial data was collected relating to flow rates and durations (see Table 4). Purified T cells and enriched PBMCs from both fresh and cryo preserved sources were used. Each experimental condition had three separate starting materials, resulting in 12 experiments, N=3 for the conditions. T cell surface markers such as activation, phenotype, and co-stimulatory markers were measured throughout the process.

Cell viability increased in the cell cultures that were exposed to 400 mL/min at the 60-minute and 120-minute conditions upon both visual and quantitative inspection. The results of these studies are summarized below in Table 4. This experiment was performed for fresh and cryo preserved T cells and PBMCs, however, the results are applicable to other source material (e.g., any cell material derived from apheresis).

Table 4 qualitatively shows cell viability response on day 8 of the experiment.

Quantitative result data is also presented herein. Peristaltic pump speed (mL/min) vs duration (minutes) is shown. Each flow rate was repeated with three different healthy donors, resulting in 9 experiments total to show reproducibility. Only at pump flow rates of 400 mL/min or more did we see a response in phenotype which is shown at 30, 60, and 120 minutes. As such, conventional pumps are unable to operate at speeds able to apply the shear forces needed to improve cell population phenotype without modification.

TABLE 4
Qualitative Phenotypic Response
Peristaltic Pump Speed vs Duration

| | 30 mins | 60 mins | 120 mins |
|---|---|---|---|
| 100 mL/min | No response | No response | No response |
| 200 mL/min | No response | Response | Response |
| 400 mL/min | Response | Response | Response |

This experiment helped establish the experimental pump speed of 400 mL/min at the four time points: 0 min (control), 30 mins, 60 mins, and 120 mins. The sampling strategy shown in Table 3 was then applied for follow-up experiments.

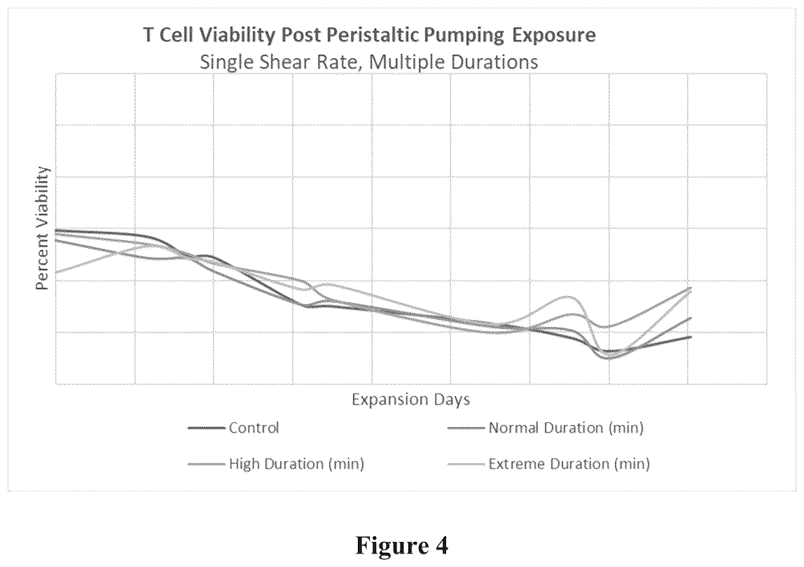

FIG. 4 shows experimental data for T cells being subjected to shear stress over various times in initial experiments for pump selection and operational parameter assessment. T cells were obtained from fresh from healthy donor samples. T cells were then activated for 48 hours. Once activated, T cells were then exposed to peristaltic pumping at 400 mL/min for either 30 minutes, 60 minutes, or 120 minutes (also see Table 4). A control was included where T cells were not subject to pumping (i.e. 0 minutes). T cells were then expanded for an additional five days for a total of eight days. A typical flow rate used in the industry of 100 mL/min was tested and showed no phenotypic response. Our experimental design included varying peristaltic pump speeds between normal (i.e. 100 mL/min), high (200 mL/min), and extreme (400 mL/min) at durations of 0 minutes, 30 minutes, 60 minutes, and 120 minutes. We also completed a screening experiment to determine if a cell phenotypic response was detected. Cell viability was selected as the response indicator. Based on our research, stimulated T cells have the ability to recover and expand when subjected to the right amount of shear stress. The results in FIG. 4 and Table 2 contributed in the decision to continue experimentation using the equipment described at 400 mL/min.

h. Cell Culture

A combination of fresh or cryo preserved T cells or PBMCs were used as the starting material for application of shear stress using a peristaltic pumping. All starting material was activated for at least 48 hours. PBMCs were activated using anti-CD3 and purified T cells were activated with anti-CD3 and anti-CD28. Once the minimum activation incubation was completed, a quarter of the cells were transferred to a new cell culture bag and kept as a control. Fresh media was be added to the bag. The remaining three quarters of the cell culture was be transferred to the mechanical force generating system and exposed to the fluidic shear stress. The peristaltic pump was operated for 30 minutes, a third of the cell culture was removed from the peristaltic pump assembly, sampled for cell counts, transferred to a cell culture bag, and fresh media was added. This process was repeated for 60 minutes and 120 minutes. Once the cells were placed in their respective cell culture bags and cell counts were taken, all culture bags were transferred to the CO2 incubator at the same time.

Twenty-four hours after the cells were seeded in the new cell culture bags, cell counts, and flow cytometry samples were assessed. Cell counts were taken throughout the expansion period until around five days later when the final cell count, and flow cytometry sample were assessed. These sample points provide a timelapse insight into the cell performance.

For fresh starting material, cell counts were taken after T cell/PBMCs selection. For cryo preserved start material, cell counts were assessed after the media exchange/wash was performed to remove DMSO. Cell counts were assessed at the start and end of activation incubation. Cell counts were assessed immediately after exposure to shear stress, 24 hours after the shear stress, and 3 days after exposure.

i. Study Design with Peristaltic Pumping

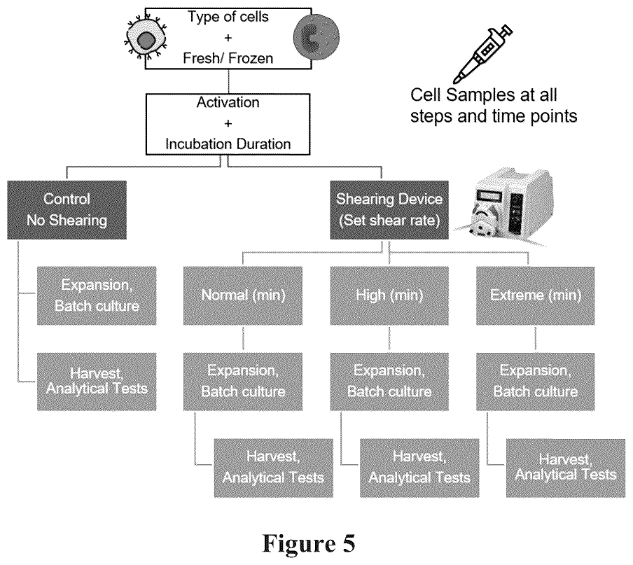

FIG. 5 illustrates a study design for peristaltic pump shearing used in the examples detailed herein. Each experiment started with either T cells or PBMCs that were fresh or cryo preserved. The cells were activated with anti-CD3 for a minimum of 48 hours. Then a quarter of the cell culture were aliquoted as the control arm. The remaining cell culture was placed in the peristaltic pump apparatus described in the previous section. The peristaltic pump recirculated the cell culture at 400 mL/min at increasing duration of 30 (normal), 60 (high), and 120 (extreme) minutes. At these time points, using the sample port, one third of the sample is aliquoted and used for expansion seed. Once the pumping has completed, all culture bags were quantum satis with fresh media and placed into the incubator. 24 hours after exposure to the peristaltic pump, analytical samples were taken, and the cell culture was returned to the incubator. Six days after pumping, the final analytical sample was taken to complete the experiment. Each condition was repeated for N=3 with different healthy donors.

TABLE 5A
Shear Force (PBMCs)

| Cell | Time | Total Shear Exposure | Average Shear Rate |
|---|---|---|---|
| Condition | (min) | (s) | (s⁻¹) |
| Fresh | 30 | 1,106,557 | 614.75 |
| | 60 | 2,213,115 | 614.75 |
| | 120 | 4,426,230 | 614.75 |
| Cryo | 30 | 1,106,557 | 614.75 |
| preserved | 60 | 2,213,115 | 614.75 |
| | 120 | 4,426,230 | 614.75 |

TABLE 5B
Shear Force (T Cells)

| Cell | Time | Total Shear Exposure | Average Shear Rate |
|---|---|---|---|
| Condition | (min) | (s) | (s⁻¹) |
| Fresh | 30 | 1,106,557 | 614.75 |
| | 60 | 2,213,115 | 614.75 |
| | 120 | 4,426,230 | 614.75 |
| Cryo | 30 | 1,106,557 | 614.75 |
| preserved | 60 | 2,213,115 | 614.75 |
| | 120 | 4,426,230 | 614.75 |

Tables 5A and 5B show the total shear exposure applied to PBMCs and T cells respectively. For the following sections, Table 5A and 5B shown how much mechanical stress was applied at different timepoints and the average rate of shear application.

The total shear exposure acting upon the cells was calculated based on clearly defined mathematical principles. Here, we used the shear rate formula for a tube under laminar flow conditions: γ=4Q/πR³
γ is the shear rate (in s⁻¹),
Q is the volumetric flow rate (in m³/s)
R is the radius of the tube (in meters)

In the experiments, the flow rate Q is constant but the duration changes. The shear itself does not change with the time duration, therefore Q (flow rate) and R (radius) remains constant. However, total volume over time changes. Therefore, to calculate shear-related effects over time, the sum of the cumulative exposure was used to quantify the total exposure. The set flow rate of the experiments was 400 mL/min. The inner diameter (ID) of the tubing used was 4.8 mm. The length of the tubing used was 45 cm. The residence time of the fluid in the tubing was 1.22 seconds and the shear exposure was 750 seconds.

$$Shear exposure per pass : Shear Exposure per Pass = Shear Rate * Residence Time$$

Shear Rate: 614,024 s⁻¹ (calculated from flow rate and tube radius)

Residence Time: 1.22 seconds (time it takes fluid to travel through the 45 cm tube)

$$Shear Exposure per Pass = 614 , 024 * 1.22 = 750 s$$

$$Number of passes in a given time : Number of passes = Total time ( seconds ) / Residence Time ( Seconds )$$

$$Total Shear Exposure : Shear exposure per pass * number of passes$$

$$Total Shear Exposure = 750 seconds * ( Total time / Residence time )$$

Therefore, the total shear exposure over the experimental conditions was determined to be:

$$30 minutes := 1 , 106 , 557 s$$

$$60 minutes := 2 , 213 , 115 s$$

$$120 minutes := 4 , 426 , 230 s$$

Although the initial experiments were performed at the 30, 60, and 120 minute intervals at 400 ml/min. Similar desirable effects of shear stress on T cells and PBMCs can be seen out of the experimental window.

### Duration of Shear Exposure

Systems that utilize a pump at a low speed for a long duration may also see desirable T cell/PBMC responses. For example, if cells in a system are recirculated constantly over an extended period of days or weeks, the continuous shear stress can cause the cells to be stimulated in the same method as described in the original work. High flow rates are not necessarily needed to show a response since the duration is much longer than the 30, 60, and 120 mins. However, the total shear stress can be calculated and compared to the total shear stress in the original experiment.

Systems that utilize various types of pumps that physically contact the cells should also result in similar responses. For example, if the system does not use a peristaltic pump, but instead, uses a centrifugal pump, the blades of the pump come into contact with the cells which can simulate their surface markers similar to the original research.

Systems that utilize non-contacting fluid methods such as a diaphragm pump can also exposure the cells to similar environments, indirectly resulting in similar responses as the original research. For example, if the cells are being moved through a system using alternating tangential flow, the cells are being moved by a difference in air pressure/vacuum, but they are also interacting with the walls of the tubing, diaphragm, and filter. This interaction is the physical stimulation that the cells need to show similar responses as seen in the original research.

To summarize, cells may be exposed in various methods such as described above which allows the physical stimulation of receptors on the cell surface. The physical stimulation, regardless of intensity and duration will result in a response that is seen in the original research.

The original research can be extrapolated to systems that have discontinuous or sustained shear exposure to cells.

### Potential Upper Limit

The Watson Marlow™ 730 Series Process pump is one of the largest, commonly used pumps in the bioprocess/pharmaceutical industry. The maximum flow rate is 3300 L/h with a 25.4 mm tubing. Converting this number to m/min=55000 m/min. The calculated shear rate is 569.78 s⁻¹.

The shear rate in the original experiment with a 4.8 mm size tubing at 400 m/min is 614.02 s⁻¹. Therefore, the experimental range covers the larger pump.

If the same pump is used with the smallest compatible tubing of 9.6 mm, then the shear at 3300 L/h (55000 m/min) is 10,533.53 s⁻¹ which should also display the cell response found in the original research.

The upper limit is reached when the total cell population is no longer viable and lysed through the physical destruction of their membrane.

j. T Cell Activation

It is known in the art that some level of T cell activation occurs in the human body. This process may be controlled by various inputs by the immune system which may allow the T cells to stay in the dormant state until they are needed. T cells in their dormant, non-activated state may be able to withstand large amounts of shear stress without affecting their cellular function or fate. Strength of T cell activation is related to the effects of fluidic shear stress as shown herein.

Although the activation pathway can be triggered in a short period of time, it takes up to 48 to 72 hours before the full effects can be measured. Exposing T cells to shear stress during the early activation periods may result in a stronger activation response.

In all experiments herein, starting materials and conditions (T cells/PBMCs*fresh/cryo preserved) a similar trend was observed when exposing the activated cell culture to 400 mL/min of pumping for the various durations. Within 24 hours, cells exposed to the extreme duration (120 mins) of pumping had the highest level of early activation marker CD69 expressed while also the lowest levels of late activation marker CD69.

Compared to the control condition, exposure to 400 mL/min for 120 minutes of peristaltic pumping has significantly magnified the early activation levels.

CD69 is known to be the early activation marker, and it is present on activated T and B cells. It is expressed upon activation via TCR or IL-2 (CD25) receptor and important for cell proliferation and survival. There is low expression in resting T cells. Its expression occurs within 3 to 12 hours in which it is elevated until 24 hours and then declines. It is known to be the earliest cell marker present during T cell activation. It is involved in T cell proliferation and functions as signal transmitting receptor. CD69 expression is required for T cell proliferation. This activation pathway is dependent on the CD3 TCR pathway. (e.g., see Shipkova, M., & Wieland, E. (2011). Surface Markers of Lymphocyte Activation and Markers of Cell Proliferation, 413(17-18), 1338-1349., the disclosure of which is hereby incorporated by reference in its entirety for all purposes.

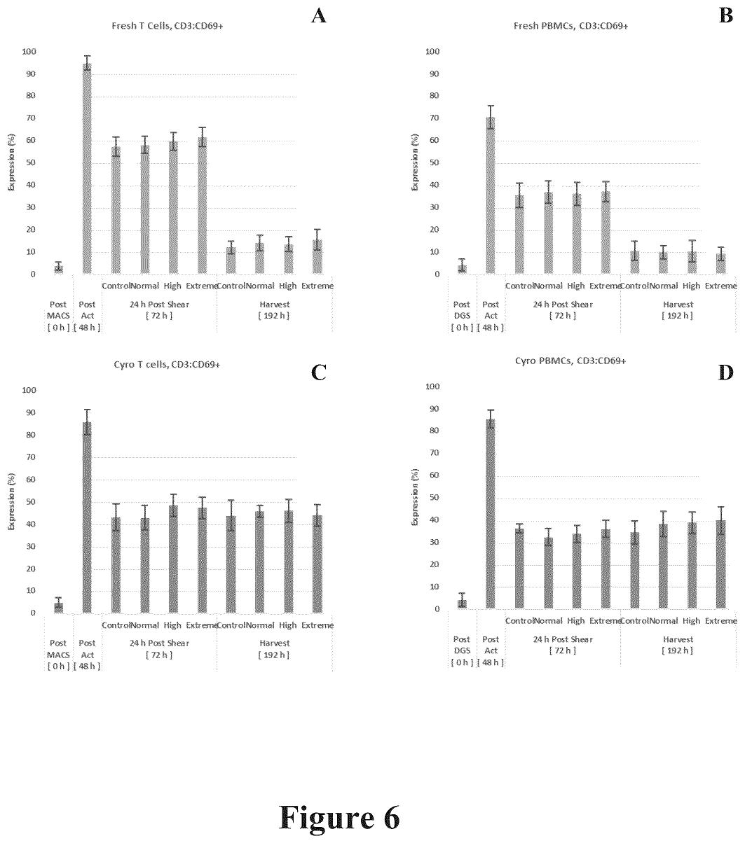

FIG. 6 shows experimental data for early T cell activation. The early T cell activation markers assessed included CD69. Peristaltic pumping flow rate was applied at 400 mL/min. For the control no flow was applied. Normal: 30 minutes, high: 60 minutes, extreme: 120 minutes of pumping. n=3 was used for all conditions.

FIG. 6, panel A shows experimental data for early T cell activation expression for fresh T cells by assessing CD69 cell surface marker expression. Magnetic activated cell sorting (MACS) was used. Percentage CD69 expression as a total of the cell population is shown on the y-axis and timepoints are shown on the x-axis. Column 1 shows pre-activation CD69 expression. Column 2 shows post activation CD69 expression after 48 hours. Column 3 shows CD69 expression after 24 hours after no shear forces having been applied and after 72 hours total (control). Columns 4, 5, and 6 show post shear force application CD69 expression after 24 hours and 72 hours total with shear forces having been applied for 30 minutes, 60 minutes, and 120 minutes respectively. We observe a clear trend of increased early T cell activation as total accumulated shear force increases over time. Column 7 shows CD69 expression after 192 hours total (control). Columns 8, 9, and 10 show post shear force application CD69 expression after 192 hours total with shear forces having been applied for 30 minutes, 60 minutes, and 120 minutes respectively. We observe a clear trend of increased early T cell activation as total accumulated shear force increases over time.

FIG. 6, panel B shows experimental data for early T cell activation expression for fresh PBMCs by assessing CD69 cell surface marker expression. DNA-gated sorting (DGS) was used. Percentage CD69 expression as a total of the cell population is shown on the y-axis and timepoints are shown on the x-axis. Column 1 shows pre-activation CD69 expression. Column 2 shows post activation CD69 expression after 48 hours. Column 3 shows CD69 expression after 24 hours after no shear forces having been applied and after 72 hours total (control). Columns 4, 5, and 6 show post shear force application CD69 expression after 24 hours and 72 hours total with shear forces having been applied for 30 minutes, 60 minutes, and 120 minutes respectively. We observe a trend of increased early T cell activation as total accumulated shear force increases over time. Column 7 shows CD69 expression after 192 hours total (control). Columns 8, 9, and 10 show post shear force application CD69 expression after 192 hours total with shear forces having been applied for 30 minutes, 60 minutes, and 120 minutes respectively. We observe a trend of increased early T cell activation as total accumulated shear force increases over time.

FIG. 6, panel C shows experimental data for early T cell activation expression for cryo preserved T cells by assessing CD69 cell surface marker expression. MACS was used. Percentage CD69 expression as a total of the cell population is shown on the y-axis and timepoints are shown on the x-axis. Column 1 shows pre-activation CD69 expression. Column 2 shows post activation CD69 expression after 48 hours. Column 3 shows CD69 expression after 24 hours after no shear forces having been applied and after 72 hours total (control). Columns 4, 5, and 6 show post shear force application CD69 expression after 24 hours and 72 hours total with shear forces having been applied for 30 minutes, 60 minutes, and 120 minutes respectively. We observe a trend of increased early T cell activation as total accumulated shear force increases over time for 24-hour post shear force application. Column 7 shows CD69 expression after 192 hours total (control). Columns 8, 9, and 10 show post shear force application CD69 expression after 192 hours total with shear forces having been applied for 30 minutes, 60 minutes, and 120 minutes respectively.

FIG. 6, panel D shows experimental data for early T cell activation expression for cryo preserved PBMCs by assessing CD69 cell surface marker expression. DGS was used. Percentage CD69 expression as a total of the cell population is shown on the y-axis and timepoints are shown on the x-axis. Column 1 shows pre-activation CD69 expression. Column 2 shows post activation CD69 expression after 48 hours. Column 3 shows CD69 expression after 24 hours after no shear forces having been applied and after 72 hours total (control). Columns 4, 5, and 6 show post shear force application CD69 expression after 24 hours and 72 hours total with shear forces having been applied for 30 minutes, 60 minutes, and 120 minutes respectively. We observe a trend of increased early T cell activation as total accumulated shear force increases over time. Column 7 shows CD69 expression after 192 hours total (control). Columns 8, 9, and 10 show post shear force application CD69 expression after 192 hours total with shear forces having been applied for 30 minutes, 60 minutes, and 120 minutes respectively. We observe a clear trend of increased early T cell activation as total accumulated shear force increases over time.

CD25 is late-stage activation marker that downregulates T cells preventing overstimulation and exhaustion. CD25 also known as the Interleukin 2 receptor (IL2RA) and is the most prominent T cell activation marker and is found on activated T cells, B cells, monocytes and macrophages. It is upregulated within 24 hours of simulation of the TCR/CD3 receptor and remains elevated for a few days.

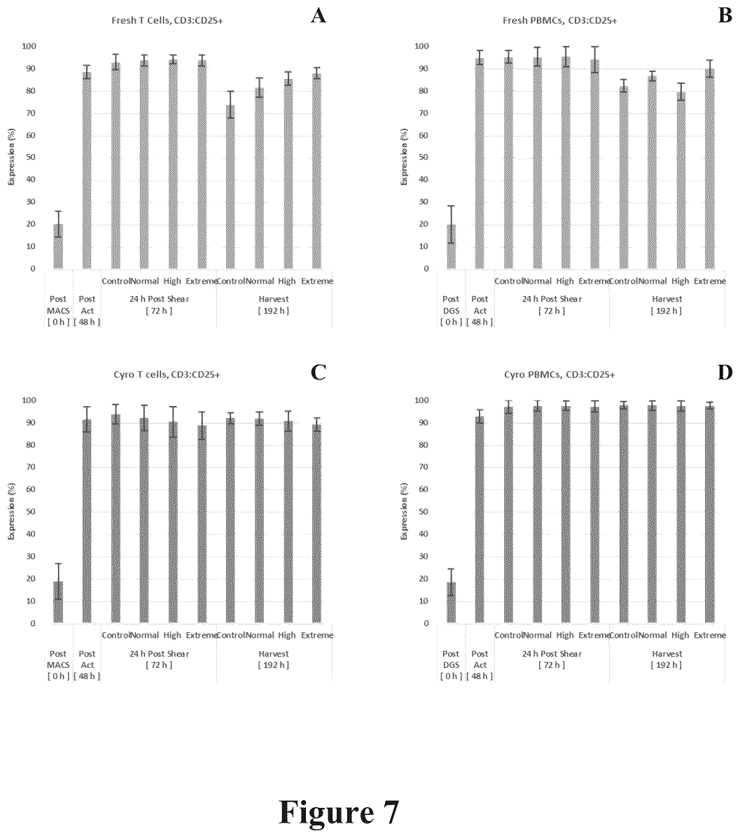

FIG. 7 shows experimental data for late T cell activation. The late T cell activation markers used included CD25. Peristaltic pumping was used at 400 mL/min. Control: no pumping, Normal: 30 minutes, high: 60 minutes, extreme: 120 minutes of pumping. n=3 for all conditions.

FIG. 7, panel A shows experimental data for early T cell activation expression for fresh T cells by assessing CD25 cell surface marker expression. MACS was used. Percentage CD25 expression as a total of the cell population is shown on the y-axis and timepoints are shown on the x-axis. Column 1 shows pre-activation CD25 expression. Column 2 shows post activation CD25 expression after 48 hours. Column 3 shows CD25 expression after 24 hours after no shear forces having been applied and after 72 hours total (control). Columns 4, 5, and 6 show post shear force application CD25 expression after 24 hours and 72 hours total with shear forces having been applied for 30 minutes, 60 minutes, and 120 minutes respectively. We observe a clear trend of increased early T cell activation as total accumulated shear force increases over time. Column 7 shows CD25 expression after 192 hours total (control). Columns 8, 9, and 10 show post shear force application CD25 expression after 192 hours total with shear forces having been applied for 30 minutes, 60 minutes, and 120 minutes respectively. We observe a profound trend of increased early T cell activation as total accumulated shear force increases over time.

FIG. 7, panel B shows experimental data for early T cell activation expression for fresh PBMCs by assessing CD25 cell surface marker expression. DGS was used. Percentage CD25 expression as a total of the cell population is shown on the y-axis and timepoints are shown on the x-axis. Column 1 shows pre-activation CD25 expression. Column 2 shows post activation CD25 expression after 48 hours. Column 3 shows CD25 expression after 24 hours after no shear forces having been applied and after 72 hours total (control). Columns 4, 5, and 6 show post shear force application CD25 expression after 24 hours and 72 hours total with shear forces having been applied for 30 minutes, 60 minutes, and 120 minutes respectively. Column 7 shows CD25 expression after 192 hours total (control). Columns 8, 9, and 10 show post shear force application CD25 expression after 192 hours total with shear forces having been applied for 30 minutes, 60 minutes, and 120 minutes respectively. We observe a trend of increased early T cell activation as total accumulated shear force increases over time.

FIG. 7, panel C shows experimental data for early T cell activation expression for cryo preserved T cells by assessing CD25 cell surface marker expression. MACS was used. Percentage CD25 expression as a total of the cell population is shown on the y-axis and timepoints are shown on the x-axis. Column 1 shows pre-activation CD25 expression. Column 2 shows post activation CD25 expression after 48 hours. Column 3 shows CD25 expression after 24 hours after no shear forces having been applied and after 72 hours total (control). Columns 4, 5, and 6 show post shear force application CD25 expression after 24 hours and 72 hours total with shear forces having been applied for 30 minutes, 60 minutes, and 120 minutes respectively. Column 7 shows CD25 expression after 192 hours total (control). Columns 8, 9, and 10 show post shear force application CD25 expression after 192 hours total with shear forces having been applied for 30 minutes, 60 minutes, and 120 minutes respectively.

FIG. 7, panel D shows experimental data for early T cell activation expression for cryo preserved PBMCs by assessing CD25 cell surface marker expression. DGS was used.

Percentage CD25 expression as a total of the cell population is shown on the y-axis and timepoints are shown on the x-axis. Column 1 shows pre-activation CD25 expression. Column 2 shows post activation CD25 expression after 48 hours. Column 3 shows CD25 expression after 24 hours after no shear forces having been applied and after 72 hours total (control). Columns 4, 5, and 6 show post shear force application CD25 expression after 24 hours and 72 hours total with shear forces having been applied for 30 minutes, 60 minutes, and 120 minutes respectively. Column 7 shows CD25 expression after 192 hours total (control). Columns 8, 9, and 10 show post shear force application CD25 expression after 192 hours total with shear forces having been applied for 30 minutes, 60 minutes, and 120 minutes respectively.

k. Memory T Cell Phenotype

The difference in phenotype composition of the T cells from a patient, at the beginning of the process detailed herein, and at the time of harvest can greatly differ depending on genetic modifications, process selection, and other factors. Here, we have demonstrated the positive impacts of applying unusually high amounts of shear force in terms of duration and total accumulated shear force.

Growth during the T cell manufacturing process may yield undesirable effects on product attributes and biological functionality. Memory T cells have four general main states: naïve, effector, central, and terminal memory. These T cells start in the naïve state and progress towards terminal memory as they are subjected to various antigens and other environmental conditions. During the T cell manufacturing process, it is common to observe T cells maturing due to activation, transduction, and expansion. We show that application of shear force using our mechanical force generation system promotes keeping the T cell population in the more desirable naïve and central memory phenotype.

Memory T cell phenotypes can differentiate into four different forms: Naïve, effector memory, central memory, and terminally differentiated cells as described above. T cell activation naturally causes cells to become more differentiated. As T cells are continuously activated, they may become exhausted and end up terminally differentiated. By the ninth day of the experiment, the extreme condition had the highest total amount of naïve and central memory T cells when compared to control. Increase in T cell activation did not encourage T cell differentiation. Research has demonstrated that human central memory or naive CD4+ and CD8+ T cells were more potent in eliminating CD19+ tumors when compared to CD19 CAR-T cells manufactured from effector memory T cells.

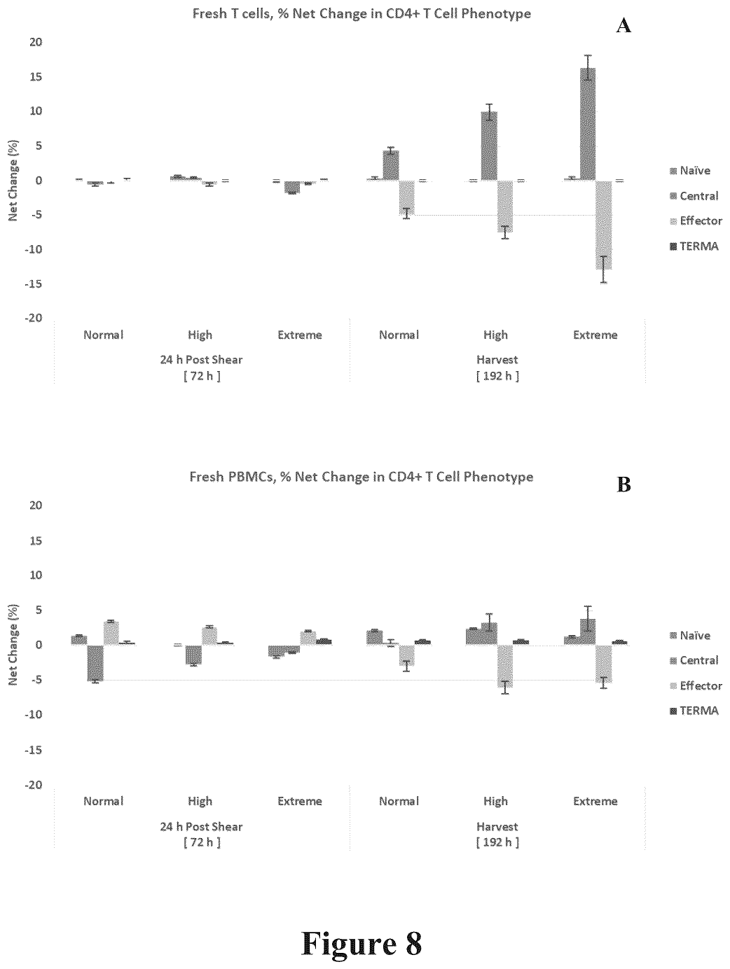

FIG. 8 shows experimental data for net change in CD3+CD4+ T cell phenotype post peristaltic pump and at harvest for fresh T cells and PBMCs as the starting material. The cell cultures were exposed to peristaltic pumping at 400 mL/min at three experimental durations: normal (30 mins), high (60 mins), and extreme (120 mins).

FIG. 8, panel A shows experimental data for net change in a CD3+CD4+ T cell phenotype post peristaltic pump and at harvest for fresh T cells by assessing CD4 cell surface marker expression. Percentage CD4 net change in expression as a total of the cell population is shown on the y-axis and timepoints are shown on the x-axis. Column set 1 shows CD4 net change in expression after 24 hours post shear force application and at 72 hours total for naïve, central, effector, and effector memory cells re-expressing CD45RA (TERMA) cells from left to right under normal shear force conditions. Column set 2 shows CD4 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. Column set 4 shows CD4 net change in expression after 192 hours total for naïve, central, effector, and TERMA cells from left to right under normal shear force conditions. Column set 5 shows CD4 net change in expression after 192 hours total for naïve, central, effector, and TERMA cells from left to right under high shear force conditions. Column set 6 shows CD4 net change in expression after 192 hours total for naïve, central, effector, and TERMA cells from left to right under extreme shear force conditions. An increasing trend in central memory T cells is clearly observable at harvest.

FIG. 8, panel B shows experimental data for net change in a CD3+CD4+ T cell phenotype post peristaltic pump and at harvest for fresh PBMCs by assessing CD4 cell surface marker expression. Percentage CD4 net change in expression as a total of the cell population is shown on the y-axis and timepoints are shown on the x-axis. Column set 1 shows CD4 net change in expression after 24 hours post shear force application and at 72 hours total for naïve, central, effector, and TERMA cells from left to right under normal shear force conditions. Column set 2 shows CD4 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. Column set 4 shows CD4 net change in expression after 192 hours total for naïve, central, effector, and TERMA cells from left to right under normal shear force conditions. Column set 5 shows CD4 net change in expression after 192 hours total for naïve, central, effector, and TERMA cells from left to right under high shear force conditions. Column set 6 shows CD4 net change in expression after 192 hours total for naïve, central, effector, and TERMA cells from left to right under extreme shear force conditions. An increasing trend in central memory T cells is clearly observable at harvest.

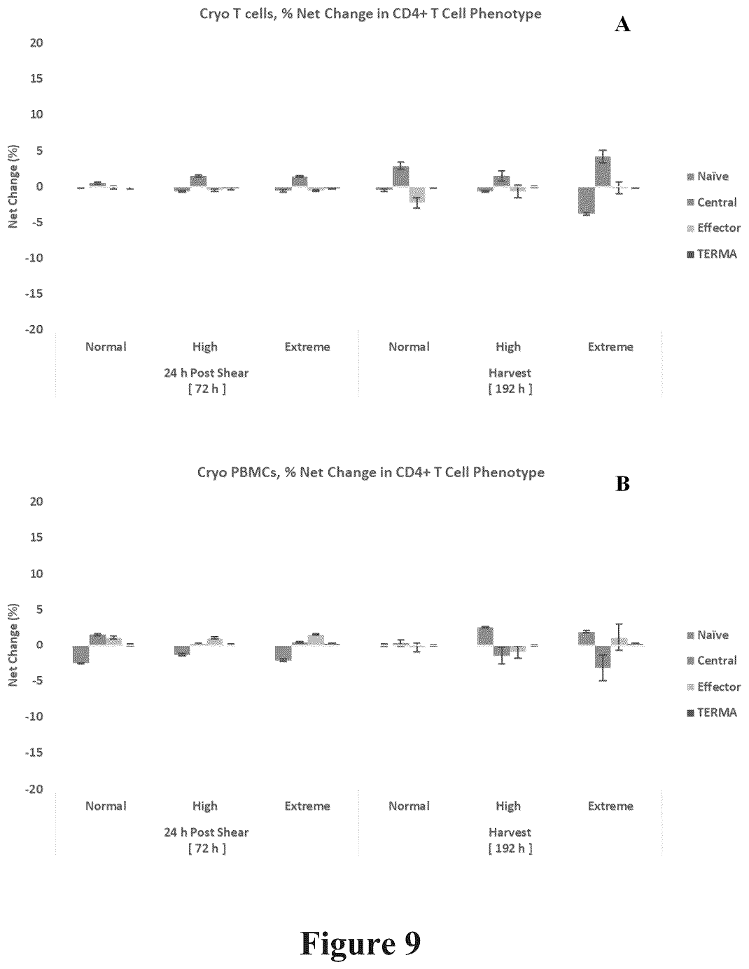

FIG. 9 shows experimental data for net change in CD3+CD4+ T cell phenotype post peristaltic pump and at harvest for cryo preserved T cells and PBMCs as the starting material.

The cell cultures were exposed to peristaltic pumping at 400 mL/min at three experimental durations: normal (30 mins), high (60 mins), and extreme (120 mins).

FIG. 9, panel A shows experimental data for net change in a CD3+CD4+ T cell phenotype post peristaltic pump and at harvest for cryo preserved T cells by assessing CD4 cell surface marker expression. Percentage CD4 net change in expression as a total of the cell population is shown on the y-axis and timepoints are shown on the x-axis. Column set 1 shows CD4 net change in expression after 24 hours post shear force application and at 72 hours total for naïve, central, effector, and TERMA cells from left to right under normal shear force conditions. Column set 2 shows CD4 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. A clear trend of increasing central memory T cells post shear was observed. Column set 4 shows CD4 net change in expression after 192 hours total for naïve, central, effector, and TERMA cells from left to right under normal shear force conditions. Column set 5 shows CD4 net change in expression after 192 hours total for naïve, central, effector, and TERMA cells from left to right under high shear force conditions. Column set 6 shows CD4 net change in expression after 192 hours total for naïve, central, effector, and TERMA cells from left to right under extreme shear force conditions. An increasing trend in central memory T cells is clearly observable at harvest.

FIG. 9, panel B shows experimental data for net change in a CD3+CD4+ T cell phenotype post peristaltic pump and at harvest for cryo preserved PBMCs by assessing CD4 cell surface marker expression. Percentage CD4 net change in expression as a total of the cell population is shown on the y-axis and timepoints are shown on the x-axis. Column set 1 shows CD4 net change in expression after 24 hours post shear force application and at 72 hours total for naïve, central, effector, and TERMA cells from left to right under normal shear force conditions. Column set 2 shows CD4 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. Column set 4 shows CD4 net change in expression after 192 hours total for naïve, central, effector, and TERMA cells from left to right under normal shear force conditions. Column set 5 shows CD4 net change in expression after 192 hours total for naïve, central, effector, and TERMA cells from left to right under high shear force conditions. Column set 6 shows CD4 net change in expression after 192 hours total for naïve, central, effector, and TERMA cells from left to right under extreme shear force conditions. An increasing trend in naïve T cells was clearly observable at harvest.

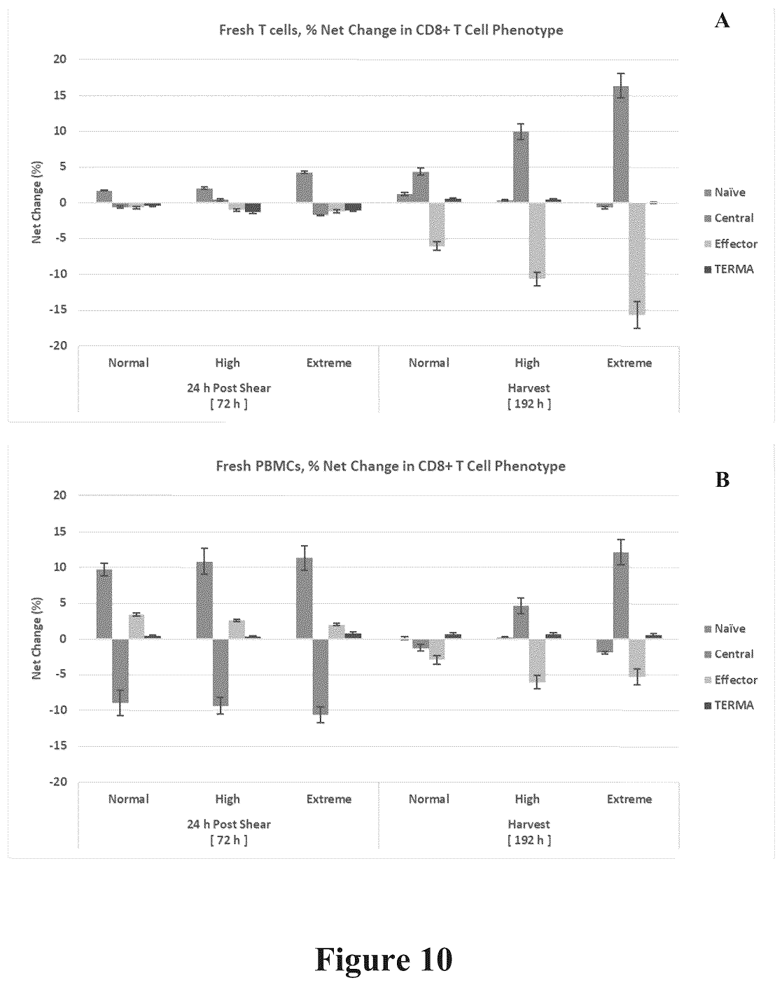

FIG. 10 shows experimental data for net change in CD3+CD8+ T cell phenotype post peristaltic pump and at harvest for fresh T cells and PBMCs as the starting material. The cell cultures were exposed to peristaltic pumping at 400 mL/min at three experimental durations: normal (30 mins), high (60 mins), and extreme (120 mins).

FIG. 10, panel A shows experimental data for net change in a CD3+CD8+ T cell phenotype post peristaltic pump and at harvest for fresh T cells by assessing CD8 cell surface marker expression. Percentage CD8 net change in expression as a total of the cell population is shown on the y-axis and timepoints are shown on the x-axis. Column set 1 shows CD8 net change in expression after 24 hours post shear force application and at 72 hours total for naïve, central, effector, and TERMA cells from left to right under normal shear force conditions. Column set 2 shows CD8 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. As application of shear force increased there was an observable increase in naïve T cells. Column set 4 shows CD8 net change in expression after 192 hours total for naïve, central, effector, and TERMA cells from left to right under normal shear force conditions. Column set 5 shows CD8 net change in expression after 192 hours total for naïve, central, effector, and TERMA cells from left to right under high shear force conditions. Column set 6 shows CD8 net change in expression after 192 hours total for naïve, central, effector, and TERMA cells from left to right under extreme shear force conditions. An increasing trend in central memory T cells is clearly observable at harvest.

FIG. 10, panel B shows experimental data for net change in a CD3+CD8+ T cell phenotype post peristaltic pump and at harvest for fresh PBMCs by assessing CD8 cell surface marker expression. Percentage CD8 net change in expression as a total of the cell population is shown on the y-axis and timepoints are shown on the x-axis. Column set 1 shows CD8 net change in expression after 24 hours post shear force application and at 72 hours total for naïve, central, effector, and TERMA cells from left to right under normal shear force conditions. Column set 2 shows CD8 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. As application of shear force increased there was an observable increase in naïve T cells. Column set 4 shows CD8 net change in expression after 192 hours total for naïve, central, effector, and TERMA cells from left to right under normal shear force conditions. Column set 5 shows CD8 net change in expression after 192 hours total for naïve, central, effector, and TERMA cells from left to right under high shear force conditions. Column set 6 shows CD8 net change in expression after 192 hours total for naïve, central, effector, and TERMA cells from left to right under extreme shear force conditions. An increasing trend in central memory T cells is clearly observable at harvest.

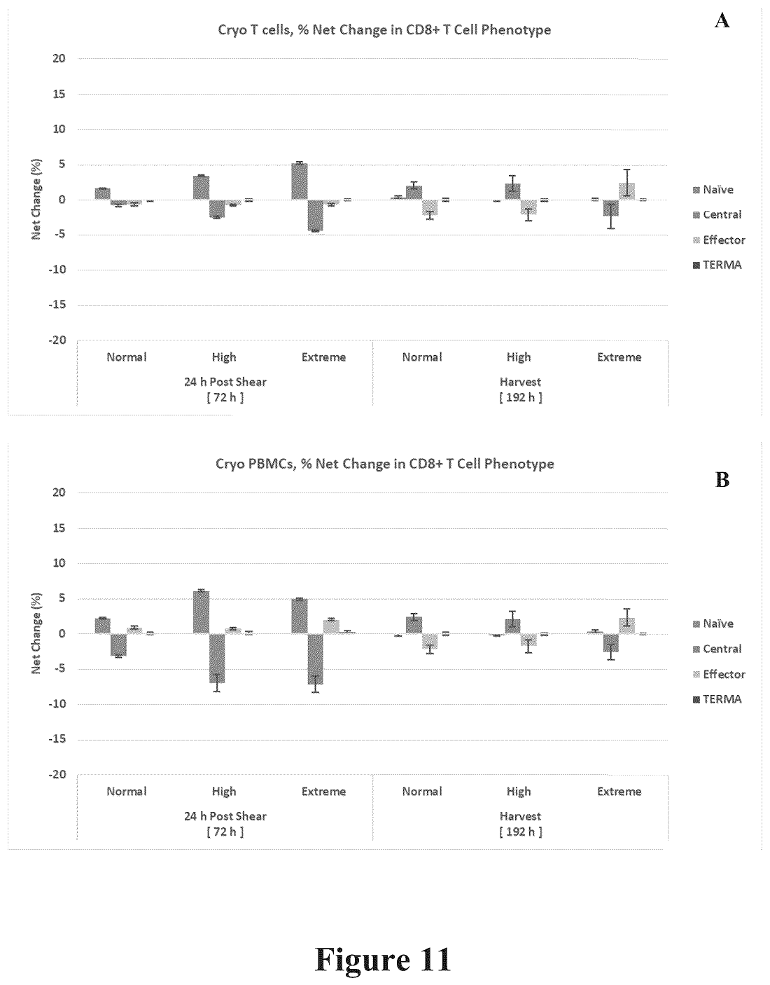

FIG. 11 shows experimental data for net change in CD3+CD8+ T cell phenotype post peristaltic pump and at harvest for cryo preserved T cells and PBMCs. The cell cultures were exposed to peristaltic pumping at 400 mL/min at three experimental durations: normal (30 mins), high (60 mins), and extreme (120 mins).

FIG. 11, panel A shows experimental data for net change in a CD3+CD8+ T cell phenotype post peristaltic pump and at harvest for cryo preserved T cells by assessing CD8 cell surface marker expression. Percentage CD8 net change in expression as a total of the cell population is shown on the y-axis and timepoints are shown on the x-axis. Column set 1 shows CD8 net change in expression after 24 hours post shear force application and at 72 hours total for naïve, central, effector, and TERMA cells from left to right under normal shear force conditions. Column set 2 shows CD8 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. As application of shear force increased there was an observable increase in naïve T cells. Column set 4 shows CD8 net change in expression after 192 hours total for naïve, central, effector, and TERMA cells from left to right under normal shear force conditions. Column set 5 shows CD8 net change in expression after 192 hours total for naïve, central, effector, and TERMA cells from left to right under high shear force conditions. Column set 6 shows CD8 net change in expression after 192 hours total for naïve, central, effector, and TERMA cells from left to right under extreme shear force conditions. An increasing trend in central memory T cells is clearly observable at harvest.

FIG. 11, panel B shows experimental data for net change in a CD3+CD8+ T cell phenotype post peristaltic pump and at harvest for cryo preserved PBMCs by assessing CD8 cell surface marker expression. Percentage CD8 net change in expression as a total of the cell population is shown on the y-axis and timepoints are shown on the x-axis. Column set 1 shows CD8 net change in expression after 24 hours post shear force application and at 72 hours total for naïve, central, effector, and TERMA cells from left to right under normal shear force conditions. Column set 2 shows CD8 net change in expression after 24 hours post shear force application and at 72 hours total for naïve, central, effector, and TERMA cells from left to right under high shear force conditions. Column set 3 shows CD4 net change in expression after 24 hours post shear force application and at 72 hours total for naïve, central, effector, and TERMA cells from left to right under extreme shear force conditions. As application of shear force increased there was an observable increase in naïve T cells. Column set 4 shows CD8 net change in expression after 192 hours total for naïve, central, effector, and TERMA cells from left to right under normal shear force conditions. Column set 5 shows CD8 net change in expression after 192 hours total for naïve, central, effector, and TERMA cells from left to right under high shear force conditions. Column set 6 shows CD8 net change in expression after 192 hours total for naïve, central, effector, and TERMA cells from left to right under extreme shear force conditions. An increasing trend in central memory T cells is clearly observable at harvest.

l. T Cell CD4:CD8 Ratio

Application of shear forces described herein lead to enhancements in CD19 CAR-T cell potency based on existing knowledge of optimal CD4+ to CD8+ ratios. This has been demonstrated by infusion to patients using defined ratios of CD8+ central memory and CD4+ T cells. A ratio of 1:1 between CD4+ and CD8+ CAR-T cells has been shown to exhibit superior persistence and stemness when compared to other ratios. We have demonstrated that the optimal ratio of 1:1 has the potential to be 5- to 100-fold more effective at a lower dose.

The normal ratio of T cell (CD3+) CD4+ to CD8+ is an important metric that may be monitored in patients. Typically, a healthy donor has a ratio of around 2.0. There should twice or more the amount of CD4 cells compared to CD8 cells. T cell activation causes both CD4 and CD8 to upregulate and proliferate. Depending on the T cell process, one of the T cell types can be favored causing the ratio to shift. The T cell process in this experiment does not influence CD4:CD8 ratios as seen in the control condition. Exposing T cells to peristaltic pumping results in an increase of CD8 T cells. The ratios decrease from 1.95, 0.75, to 0.72 as the pumping duration increases from 30 mins, 60 mins, to 120 mins respectively. Research has shown that a T cell population at the experiment with a CD4:CD8 ratio closer to 1.0 has a better clinical response.

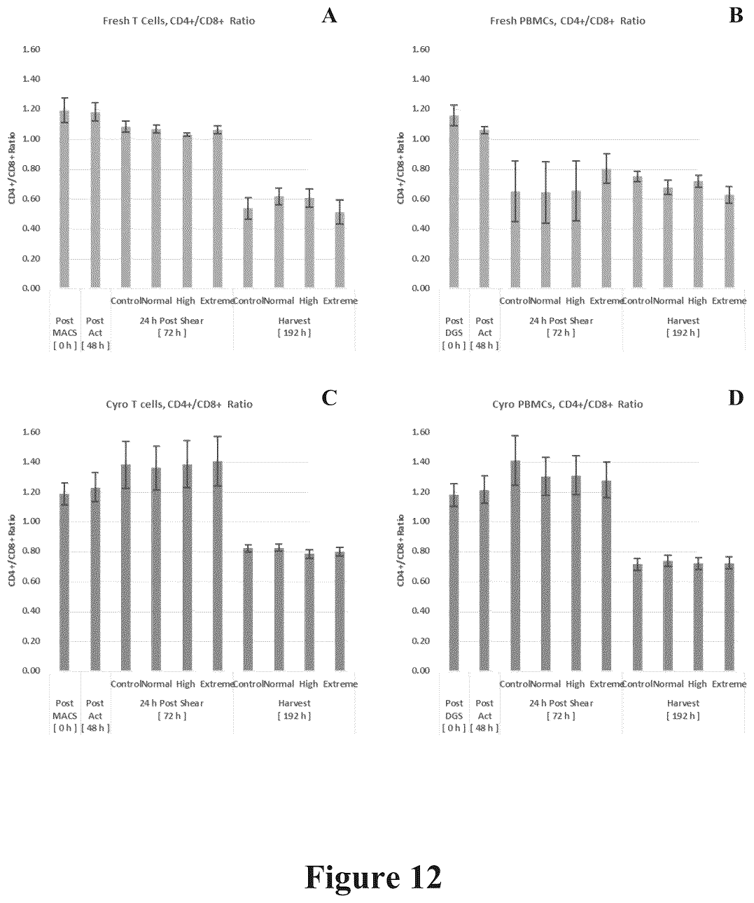

FIG. 12 shows experimental data for T cell CD4+/CD8+ ratio. Peristaltic pumping was kept at 400 mL/min for fresh and cryo preserved T cells and PBMCs. Control: no pumping, Normal: 30 mins, high: 60 mins, extreme: 120 mins of pumping. n=3 for all conditions.

FIG. 12, panel A shows experimental data for CD4+/CD8+ ratio for fresh T cells by assessing CD4 and CD8 cell surface marker expression. MACS was used. The ratio of CD4 and CD8 expression compared to a total of the cell population is shown on the y-axis and timepoints are shown on the x-axis. Column 1 shows pre-activation CD4+/CD8+ ratio expression. Column 2 shows post activation CD4+/CD8+ ratio expression after 48 hours. Column 3 shows CD4+/CD8+ ratio expression after 24 hours after no shear forces having been applied and after 72 hours total (control). Columns 4, 5, and 6 show post shear force application CD4+/CD8+ ratio expression after 24 hours and 72 hours total with shear forces having been applied for 30 minutes, 60 minutes, and 120 minutes respectively. We observe a clear trend of CD4+/CD8+ ratio converging to 1:1 over time. Column 7 shows CD4+/CD8+ ratio expression after 192 hours total (control). Columns 8, 9, and 10 show post shear force application CD4+/CD8+ ratio expression after 192 hours total with shear forces having been applied for 30 minutes, 60 minutes, and 120 minutes respectively.

FIG. 12, panel B shows experimental data for CD4+/CD8+ ratio for fresh PBMCs by assessing CD4 and CD8 cell surface marker expression. DGS was used. The ratio of CD4 and CD8 expression compared to a total of the cell population is shown on the y-axis and timepoints are shown on the x-axis. Column 1 shows pre-activation CD4+/CD8+ ratio expression. Column 2 shows post activation CD4+/CD8+ ratio expression after 48 hours. Column 3 shows CD4+/CD8+ ratio expression after 24 hours after no shear forces having been applied and after 72 hours total (control). Columns 4, 5, and 6 show post shear force application CD4+/CD8+ ratio expression after 24 hours and 72 hours total with shear forces having been applied for 30 minutes, 60 minutes, and 120 minutes respectively. Column 7 shows CD4+/CD8+ ratio expression after 192 hours total (control). Columns 8, 9, and 10 show post shear force application CD4+/CD8+ ratio expression after 192 hours total with shear forces having been applied for 30 minutes, 60 minutes, and 120 minutes respectively.

FIG. 12, panel C shows experimental data for CD4+/CD8+ ratio for cryo preserved T cells by assessing CD4 and CD8 cell surface marker expression. MACS was used. The ratio of CD4 and CD8 expression compared to a total of the cell population is shown on the y-axis and timepoints are shown on the x-axis. Column 1 shows pre-activation CD4+/CD8+ ratio expression. Column 2 shows post activation CD4+/CD8+ ratio expression after 48 hours. Column 3 shows CD4+/CD8+ ratio expression after 24 hours after no shear forces having been applied and after 72 hours total (control). Columns 4, 5, and 6 show post shear force application CD4+/CD8+ ratio expression after 24 hours and 72 hours total with shear forces having been applied for 30 minutes, 60 minutes, and 120 minutes respectively. Column 7 shows CD4+/CD8+ ratio expression after 192 hours total (control). Columns 8, 9, and 10 show post shear force application CD4+/CD8+ ratio expression after 192 hours total with shear forces having been applied for 30 minutes, 60 minutes, and 120 minutes respectively.

FIG. 12, panel D shows experimental data for CD4+/CD8+ ratio for cryo preserved PBMCs by assessing CD4 and CD8 cell surface marker expression. DGS was used. The ratio of CD4 and CD8 expression compared to a total of the cell population is shown on the y-axis and timepoints are shown on the x-axis. Column 1 shows pre-activation CD4+/CD8+ ratio expression. Column 2 shows post activation CD4+/CD8+ ratio expression after 48 hours. Column 3 shows CD4+/CD8+ ratio expression after 24 hours after no shear forces having been applied and after 72 hours total (control). Columns 4, 5, and 6 show post shear force application CD4+/CD8+ ratio expression after 24 hours and 72 hours total with shear forces having been applied for 30 minutes, 60 minutes, and 120 minutes respectively. A visible trend toward a CD4+/CD8+ ratio of 1:1 was observed. Column 7 shows CD4+/CD8+ ratio expression after 192 hours total (control). Columns 8, 9, and 10 show post shear force application CD4+/CD8+ ratio expression after 192 hours total with shear forces having been applied for 30 minutes, 60 minutes, and 120 minutes respectively.

m. T Cell PD-1 Expression

PD-1, also known as CD279 or programmed cell death 1, is a central inhibitory receptor regulating CD8 T cell exhaustion. PD-1 plays an inhibitory role in the naïve-to-effector CD8 T cell transition and the PD-1 pathway can be modulated during T cell differentiation. Infection results in activation of pathogen-specific naïve CD8+ T cells, followed by clonal expansion, differentiation into effector CD8+ T cells, then apoptosis of a majority (90-95%) of effector CD8+ T cells. The remaining effector CD8+ T cells differentiate into memory T cells to provide long-term protective immunity. PD-1 may also be expressed during the early phase of T cell activation when naïve CD8+ T cells differentiate into effector cells. During activation, PD-1, CD25, and CD69 are also rapidly up-regulated. PD-1 is upregulated by TCR signaling via T cell activation. Excessive and continuous T cell activation can cause terminal differentiation and apoptosis which forces the negative feedback system to optimize T cell effector and memory responses.

24 hour post peristaltic pumping at 400 mL/min, all experimental conditions had elevated levels of PD-1 expression, correlating to the upregulated CD69+ early activation marker. PD-1 expression at the end of the experiment was the lowest in the extreme condition (120 mins) also correlating to the late activation marker CD25. Increasing levels of T cell activation does not uncontrollably upregulate PD-1 expression.

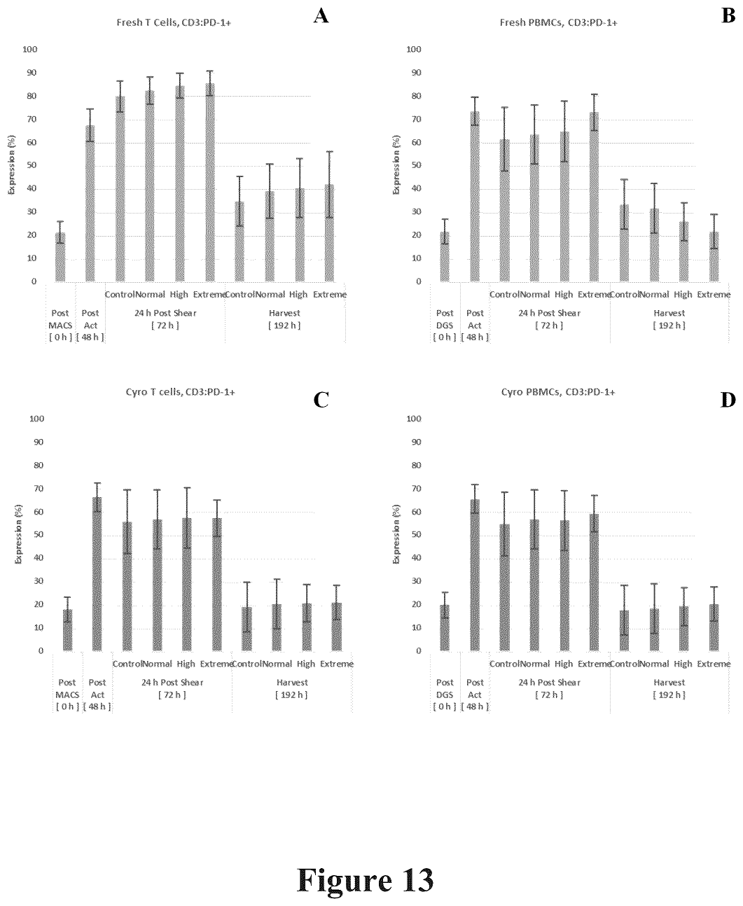

FIG. 13 shows experimental data for programmed cell death protein 1 marker CD3+PD-1+. Peristaltic pumping was kept at 400 mL/min. Control: no pumping, Normal: 30 mins, high: 60 mins, extreme: 120 mins of pumping. (n=3 for all conditions).

FIG. 13, panel A shows experimental data for PD-1+ expression for fresh T cells by assessing PD-1 cell surface marker expression. MACS was used. Percentage PD-1 expression as a total of the cell population is shown on the y-axis and timepoints are shown on the x-axis. Column 1 shows pre-activation PD-1 expression. Column 2 shows post activation PD-1 expression after 48 hours. Column 3 shows PD-1 expression after 24 hours after no shear forces having been applied and after 72 hours total (control). Columns 4, 5, and 6 show post shear force application PD-1 expression after 24 hours and 72 hours total with shear forces having been applied for 30 minutes, 60 minutes, and 120 minutes respectively. We observe a clear trend of increased PD-1 expression as total accumulated shear force increases over time. Column 7 shows PD-1 expression after 192 hours total (control). Columns 8, 9, and 10 show post shear force application PD-1 expression after 192 hours total with shear forces having been applied for 30 minutes, 60 minutes, and 120 minutes respectively.

FIG. 13, panel B shows experimental data for PD-1+ expression for fresh PBMCs by assessing PD-1 cell surface marker expression. DGS was used. Percentage PD-1 expression as a total of the cell population is shown on the y-axis and timepoints are shown on the x-axis. Column 1 shows pre-activation PD-1 expression. Column 2 shows post activation PD-1 expression after 48 hours. Column 3 shows PD-1 expression after 24 hours after no shear forces having been applied and after 72 hours total (control). Columns 4, 5, and 6 show post shear force application PD-1 expression after 24 hours and 72 hours total with shear forces having been applied for 30 minutes, 60 minutes, and 120 minutes respectively. Column 7 shows PD-1 expression after 192 hours total (control). Columns 8, 9, and 10 show post shear force application PD-1 expression after 192 hours total with shear forces having been applied for 30 minutes, 60 minutes, and 120 minutes respectively.

FIG. 13, panel C shows experimental data for PD-1+ expression for cryo preserved T cells by assessing PD-1 cell surface marker expression. Magnetic activated cell sorting (MACS) was used. Percentage PD-1 expression as a total of the cell population is shown on the y-axis and timepoints are shown on the x-axis. Column 1 shows pre-activation PD-1 expression. Column 2 shows post activation PD-1 expression after 48 hours. Column 3 shows PD-1 expression after 24 hours after no shear forces having been applied and after 72 hours total (control). Columns 4, 5, and 6 show post shear force application PD-1 expression after 24 hours and 72 hours total with shear forces having been applied for 30 minutes, 60 minutes, and 120 minutes respectively. Column 7 shows PD-1 expression after 192 hours total (control). Columns 8, 9, and 10 show post shear force application PD-1 expression after 192 hours total with shear forces having been applied for 30 minutes, 60 minutes, and 120 minutes respectively.

FIG. 13, panel D shows experimental data for PD-1+ expression for cryo preserved PBMCs by assessing PD-1 cell surface marker expression. DNA-gated sorting (DGS) was used. Percentage PD-1 expression as a total of the cell population is shown on the y-axis and timepoints are shown on the x-axis. Column 1 shows pre-activation PD-1 expression. Column 2 shows post activation PD-1 expression after 48 hours. Column 3 shows PD-1 expression after 24 hours after no shear forces having been applied and after 72 hours total (control). Columns 4, 5, and 6 show post shear force application PD-1 expression after 24 hours and 72 hours total with shear forces having been applied for 30 minutes, 60 minutes, and 120 minutes respectively. Column 7 shows PD-1 expression after 192 hours total (control). Columns 8, 9, and 10 show post shear force application PD-1 expression after 192 hours total with shear forces having been applied for 30 minutes, 60 minutes, and 120 minutes respectively.

n. T Cell CD27/CD28 Expression

CD28 is also known as T cell-specific surface glycoprotein and it is found on mature CD3+ thymocytes, most peripheral T cells, and plasma cells. Co-stimulation of T cell proliferation and cytokine production by binding to its ligands CD80 or CD86, co-stimulates T cell effector function and T cell-dependent antibody production. CD28 is found on 95% of CD4+ T cells and about 50% of CD8+ T cells. T cells stimulated by both CD3 and CD28 have enhanced cell metabolism, increased IL-2 and other cytokine production by 5 to 50-fold. CD28 is expressed by both CD4+ and CD8+ cytotoxic cells, and activation through the previously described pathway can augment their cytotoxic potential while also upregulated cytokines such as IL-2, IFN-g, TNF-α, and TNF-b.

Proper T cell activation requires co-stimulation of both the CD27 and CD28 receptors. CD27 marker is associated with T cell differentiation. CD27 also known as tumor necrosis factor receptor superfamily member. This marker is found on most T cells, memory B cells, and NK cells. It is a receptor for CD70/CD27L and may play a role in survival of activated T cells and apoptosis. It is involved in regulation of B cell activation and immunoglobulin synthesis.

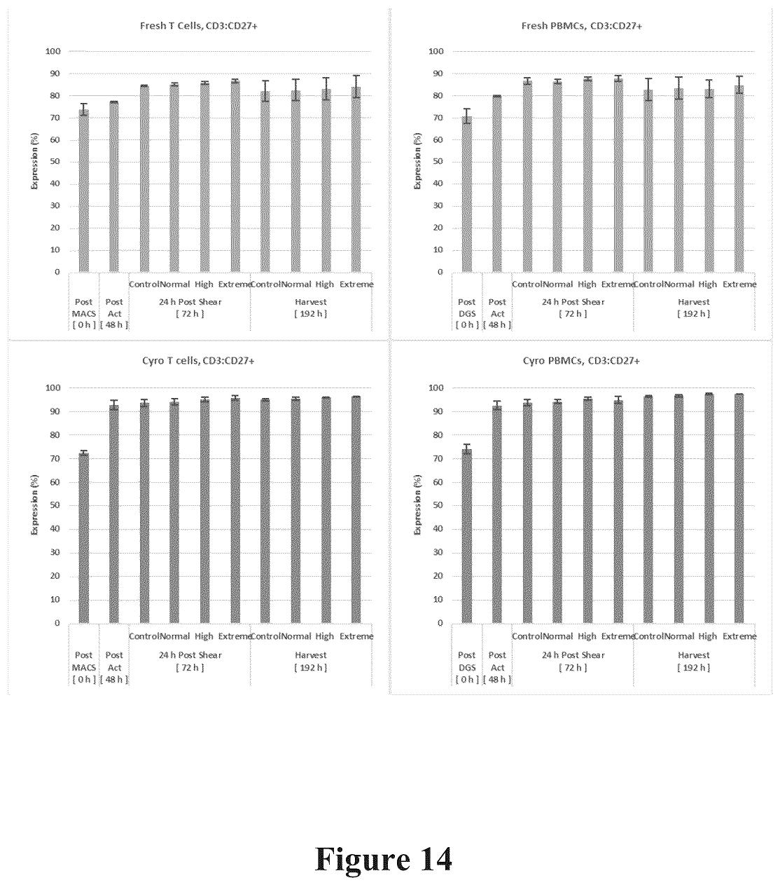

CD27 marker promotes survival of activated T cells and complements the CD28 costimulatory marker in effector T cells. The expression of this marker dictates the magnitude of primary and memory T cell responses while also promoting the survival of activated T cells through rounds of cell division. CD28 receptor needs to be stimulated simultaneously with the CD3/TCR receptor in order to ensure full T cell activation. FIG. 14 illustrates CD28 expression levels through the T cell process. At the end of the experiment, both costimulatory markers are expressed in levels similar to the control condition. Peristaltic pumping does not damage or interfere with the CD27 and CD28 receptors.

FIG. 14 shows experimental data for co-stimulatory marker CD3+CD27+. Peristaltic pumping was kept at 400 mL/min. Control: no pumping, Normal: 30 mins, high: 60 mins, extreme: 120 mins of pumping. n=3 for all conditions.

FIG. 14, panel A shows experimental data for T cell CD27+ expression for fresh T cells by assessing CD27 cell surface marker expression. MACS was used. Percentage CD27 expression as a total of the cell population is shown on the y-axis and timepoints are shown on the x-axis. Column 1 shows pre-activation CD27 expression. Column 2 shows post activation CD27 expression after 48 hours. Column 3 shows CD27 expression after 24 hours after no shear forces having been applied and after 72 hours total (control). Columns 4, 5, and 6 show post shear force application CD27 expression after 24 hours and 72 hours total with shear forces having been applied for 30 minutes, 60 minutes, and 120 minutes respectively. Column 7 shows CD27 expression after 192 hours total (control). Columns 8, 9, and 10 show post shear force application CD28 expression after 192 hours total with shear forces having been applied for 30 minutes, 60 minutes, and 120 minutes respectively.

FIG. 14, panel B shows experimental data for CD27 for fresh T cells by assessing CD27 cell surface marker expression. DGS was used. Percentage CD27 expression as a total of the cell population is shown on the y-axis and timepoints are shown on the x-axis. Column 1 shows pre-activation CD27 expression. Column 2 shows post activation CD27 expression after 48 hours. Column 3 shows CD27 expression after 24 hours after no shear forces having been applied and after 72 hours total (control). Columns 4, 5, and 6 show post shear force application CD27 expression after 24 hours and 72 hours total with shear forces having been applied for 30 minutes, 60 minutes, and 120 minutes respectively. Column 7 shows CD27 expression after 192 hours total (control). Columns 8, 9, and 10 show post shear force application CD27 expression after 192 hours total with shear forces having been applied for 30 minutes, 60 minutes, and 120 minutes respectively.

FIG. 14, panel C shows experimental data for CD27 expression for cryo preserved T cells by assessing CD27 cell surface marker expression. MACS was used. Percentage CD27 expression as a total of the cell population is shown on the y-axis and timepoints are shown on the x-axis. Column 1 shows pre-activation CD27 expression. Column 2 shows post activation CD27 expression after 48 hours. Column 3 shows CD27 expression after 24 hours after no shear forces having been applied and after 72 hours total (control). Columns 4, 5, and 6 show post shear force application CD27 expression after 24 hours and 72 hours total with shear forces having been applied for 30 minutes, 60 minutes, and 120 minutes respectively. Column 7 shows CD27 expression after 192 hours total (control). Columns 8, 9, and 10 show post shear force application CD27 expression after 192 hours total with shear forces having been applied for 30 minutes, 60 minutes, and 120 minutes respectively.

FIG. 14, panel D shows experimental data for early CD27 expression for cryo preserved PBMCs by assessing CD27 cell surface marker expression. DNA-gated sorting (DGS) was used. Percentage CD27 expression as a total of the cell population is shown on the y-axis and timepoints are shown on the x-axis. Column 1 shows pre-activation CD27 expression. Column 2 shows post activation CD27 expression after 48 hours. Column 3 shows CD27 expression after 24 hours after no shear forces having been applied and after 72 hours total (control). Columns 4, 5, and 6 show post shear force application CD27 expression after 24 hours and 72 hours total with shear forces having been applied for 30 minutes, 60 minutes, and 120 minutes respectively. Column 7 shows CD27 expression after 192 hours total (control). Columns 8, 9, and 10 show post shear force application CD27 expression after 192 hours total with shear forces having been applied for 30 minutes, 60 minutes, and 120 minutes respectively.

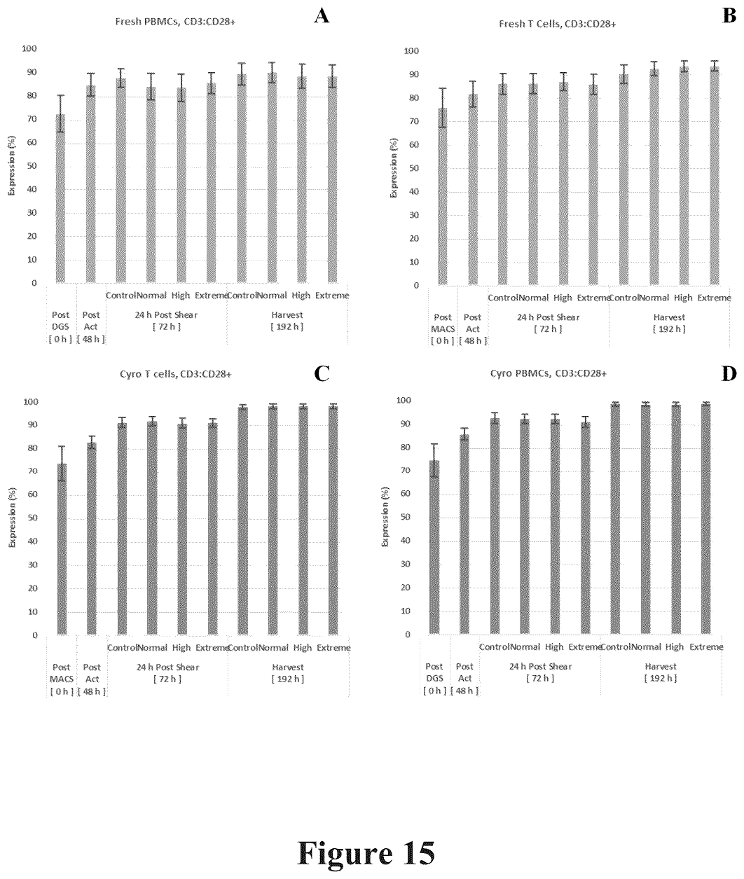

FIG. 15 shows experimental data for co-stimulatory marker CD3+CD28+. Peristaltic pumping was kept at 400 mL/min. Control: no pumping, Normal: 30 mins, high: 60 mins, extreme: 120 mins of pumping. n=3 for all conditions.

FIG. 15, panel A shows experimental data for T cell CD28 expression for PBMCs by assessing CD28 cell surface marker expression. DGS was used. Percentage CD28 expression as a total of the cell population is shown on the y-axis and timepoints are shown on the x-axis. Column 1 shows pre-activation CD28 expression. Column 2 shows post activation CD28 expression after 48 hours. Column 3 shows CD28 expression after 24 hours after no shear forces having been applied and after 72 hours total (control). Columns 4, 5, and 6 show post shear force application CD28 expression after 24 hours and 72 hours total with shear forces having been applied for 30 minutes, 60 minutes, and 120 minutes respectively. Column 7 shows CD28 expression after 192 hours total (control). Columns 8, 9, and 10 show post shear force application CD28 expression after 192 hours total with shear forces having been applied for 30 minutes, 60 minutes, and 120 minutes respectively.

FIG. 15, panel B shows experimental data for CD28 expression for fresh T cells by assessing CD28 cell surface marker expression. MACS was used. Percentage CD28 expression as a total of the cell population is shown on the y-axis and timepoints are shown on the x-axis. Column 1 shows pre-activation CD28 expression. Column 2 shows post activation CD28 expression after 48 hours. Column 3 shows CD28 expression after 24 hours after no shear forces having been applied and after 72 hours total (control). Columns 4, 5, and 6 show post shear force application CD28 expression after 24 hours and 72 hours total with shear forces having been applied for 30 minutes, 60 minutes, and 120 minutes respectively. Column 7 shows CD28 expression after 192 hours total (control). Columns 8, 9, and 10 show post shear force application CD28 expression after 192 hours total with shear forces having been applied for 30 minutes, 60 minutes, and 120 minutes respectively. A trend in increasing CD28 expression was observable as shear force application is increased.

FIG. 15, panel C shows experimental data for CD28 expression for cryo preserved T cells by assessing CD28 cell surface marker expression. MACS was used. Percentage CD28 expression as a total of the cell population is shown on the y-axis and timepoints are shown on the x-axis. Column 1 shows pre-activation CD28 expression. Column 2 shows post activation CD28 expression after 48 hours. Column 3 shows CD28 expression after 24 hours after no shear forces having been applied and after 72 hours total (control). Columns 4, 5, and 6 show post shear force application CD28 expression after 24 hours and 72 hours total with shear forces having been applied for 30 minutes, 60 minutes, and 120 minutes respectively. Column 7 shows CD28 expression after 192 hours total (control). Columns 8, 9, and 10 show post shear force application CD28 expression after 192 hours total with shear forces having been applied for 30 minutes, 60 minutes, and 120 minutes respectively.

FIG. 15, panel D shows experimental data for CD28 expression for cryo preserved PBMCs by assessing CD28 cell surface marker expression. DNA-gated sorting (DGS) was used. Percentage CD28 expression as a total of the cell population is shown on the y-axis and timepoints are shown on the x-axis. Column 1 shows pre-activation CD28 expression. Column 2 shows post activation CD28 expression after 48 hours. Column 3 shows CD28 expression after 24 hours after no shear forces having been applied and after 72 hours total (control). Columns 4, 5, and 6 show post shear force application CD28 expression after 24 hours and 72 hours total with shear forces having been applied for 30 minutes, 60 minutes, and 120 minutes respectively. Column 7 shows CD28 expression after 192 hours total (control). Columns 8, 9, and 10 show post shear force application CD28 expression after 192 hours total with shear forces having been applied for 30 minutes, 60 minutes, and 120 minutes respectively.

o. T Cells, B Cells, and NK Cells Populations

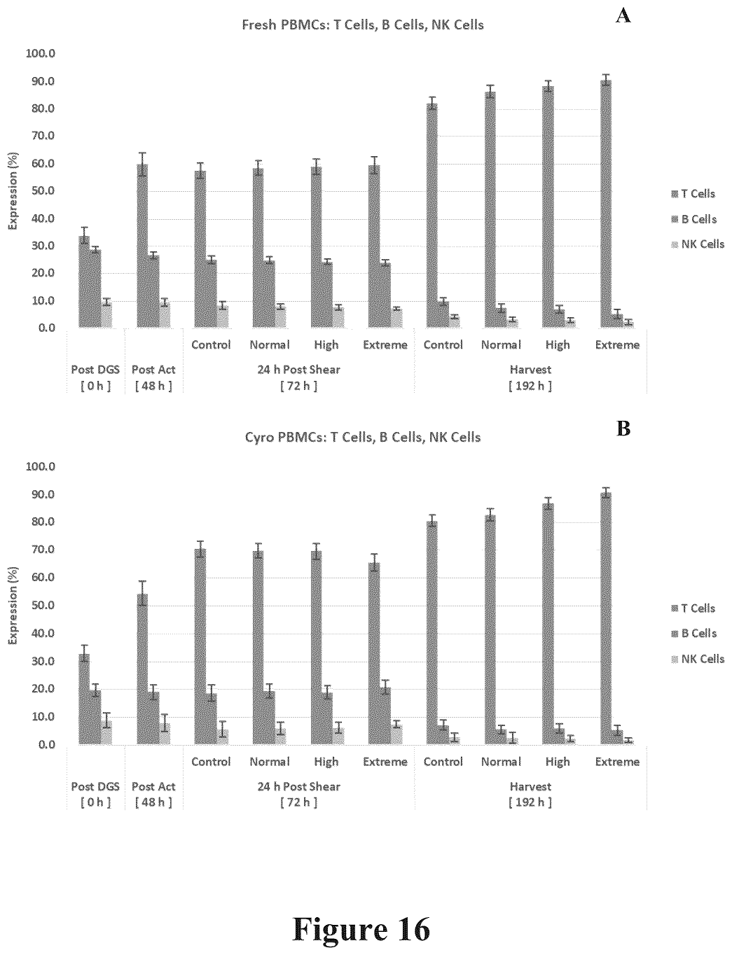

FIG. 16 shows experimental data for relative expression of T cells, B cells, and NK cells populations for fresh T cells and PBMCs. Peristaltic pumping was kept at 400 mL/min.

Control: no pumping, Normal: 30 mins, high: 60 mins, extreme: 120 mins of pumping. n=3 was used for all conditions.

FIG. 16, panel A shows experimental data for net change in a T cells, B cells, and NK cells populations post peristaltic pump and at harvest for fresh T cells by assessing expression of the markers described in the examples section herein (e.g., CD3, CD19, and CD56). Percentage net change in expression as a total of the cell population is shown on the y-axis and timepoints are shown on the x-axis. Column set 1 shows net change in expression after before shear force application and after DGS for T cells, B cells, and NK cells from left to right. Column set 2 shows net change in expression after 48 hours post activation for T cells, B cells, and NK cells from left to right. Column set 3 shows net change in expression after 24 hours post no shear force application and at 72 hours total for T cells, B cells, and NK cells from left to right. Column set 4 shows net change in expression after 24 hours post shear force application and at 72 hours total for T cells, B cells, and NK cells from left to right under normal shear force conditions. Column set 5 shows net change in expression after 24 hours post shear force application and at 72 hours total for T cells, B cells, and NK cells from left to right under high shear force conditions. Column set 6 shows net change in expression after 24 hours post shear force application and at 72 hours total for T cells, B cells, and NK cells from left to right under extreme shear force conditions. Column set 7 shows net change in expression after 192 hours total with no shear force application and at harvest for T cells, B cells, and NK cells from left to right. Column set 8 shows net change in expression after 192 hours total with shear force application and at harvest for T cells, B cells, and NK cells from left to right for normal shear force conditions. Column set 9 shows net change in expression after 192 hours total with shear force application and at harvest for T cells, B cells, and NK cells from left to right for high shear force conditions. Column set 10 shows net change in expression after 192 hours total with shear force application and at harvest for T cells, B cells, and NK cells from left to right for extreme shear force conditions. An increasing trend in total T cells is observable at harvest.

FIG. 16, panel B shows experimental data for net change in a T cells, B cells, and NK cells populations post peristaltic pump and at harvest for cryo preserved PMBCs assessing expression of the markers described in the examples section herein (e.g., CD3, CD19, and CD56). Percentage net change in expression as a total of the cell population is shown on the y-axis and timepoints are shown on the x-axis. Column set 1 shows net change in expression after before shear force application and after DGS for T cells, B cells, and NK cells from left to right.

Column set 2 shows net change in expression after 48 hours post activation for T cells, B cells, and NK cells from left to right. Column set 3 shows net change in expression after 24 hours post no shear force application and at 72 hours total for T cells, B cells, and NK cells from left to right. Column set 4 shows net change in expression after 24 hours post shear force application and at 72 hours total for T cells, B cells, and NK cells from left to right under normal shear force conditions. Column set 5 shows net change in expression after 24 hours post shear force application and at 72 hours total for T cells, B cells, and NK cells from left to right under high shear force conditions. Column set 6 shows net change in expression after 24 hours post shear force application and at 72 hours total for T cells, B cells, and NK cells from left to right under extreme shear force conditions. Column set 7 shows net change in expression after 192 hours total with no shear force application and at harvest for T cells, B cells, and NK cells from left to right. Column set 8 shows net change in expression after 192 hours total with shear force application and at harvest for T cells, B cells, and NK cells from left to right for normal shear force conditions. Column set 9 shows net change in expression after 192 hours total with shear force application and at harvest for T cells, B cells, and NK cells from left to right for high shear force conditions. Column set 10 shows net change in expression after 192 hours total with shear force application and at harvest for T cells, B cells, and NK cells from left to right for extreme shear force conditions. An increasing trend in total T cells is observable at harvest.

p. Interleukin-2 Concentration

Interleukin-2 is a cytokine that plays a key role in activation, survival, expansion, and function of T cells by controlling the differentiation and homeostasis of T cells. IL-2 is critical for the development and proliferation of T cells. The presence of IL-2 will upregulate effector T cells, downregulate central memory T cells, and maintain regulatory T cells. Generally, IL-2 is used during initial T cell activation and proliferation. IL-2 levels may be depleted, T cells have the ability to produce and secrete their own IL-2 starting a positive feedback loop. As IL-2 is secreted into the surrounding environment, T cell proliferation continues while prolonging differentiation to terminated T cells.

The binding of IL-2 to the IL-2R receptor provides the necessary signal to stimulate T cells from the G1 to the S phase of the cell cycle. This mechanism is the primary regulatory mechanism of T cell proliferation. Triggering Cd4+ T cells by antigenic or mitogenic stimuli induces secretion of IL-2. IL-2 released by the cells can have an autocrine effect in which the IL-2 can bind to neighboring cells' IL-2R receptors and promoting proliferation. Secreted IL-2 can also have a paracrine effect in which it binds and induces the growth of other cells, such as CD8+ T cells, B cells, and NK cells. The previously mentioned cells have IL-2R receptors, but they cannot synthesize their own IL-2. IL-2 can also promote the growth of memory T cells.

Both T cell and PBMCs in all conditions demonstrate a similar trend in which initial IL-2 levels in suspension are plentiful due to IL-2 present in the media. 48 hours later, IL-2 is consumed by the T cells during activation. At the end of the experiment, IL-2 levels present in the media are significantly high in all conditions exposed to pumping. The extreme condition (e.g., 120 minutes) has the highest level of IL-2 in solution, potentially indicating that the T cells in that experimental condition are continuing to proliferate and secrete the protein.

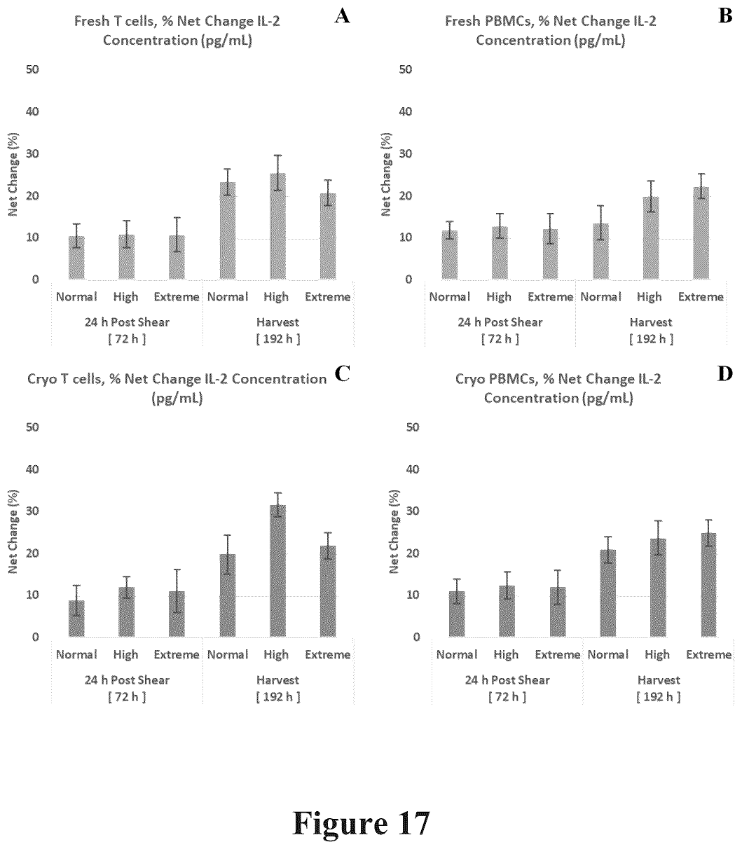

FIG. 17 shows experimental data for net IL-2 concentration change. Normalized to control. Normal: 30 mins, high: 60 mins, extreme: 120 mins of pumping. n=3 was used for all conditions.

FIG. 17, panel A shows experimental data for IL-2 expression for fresh T cells by assessing IL-2 marker. Percentage IL-2 expression as a total of the cell population is shown on the y-axis and timepoints are shown on the x-axis. Columns 1, 2, and 3 show post shear force application IL-2 expression after 24 hours and 72 hours total with shear forces having been applied for 30 minutes, 60 minutes, and 120 minutes respectively. Columns 4, 5, and 6 show post shear force application IL-2 expression after 192, at harvest, hours total with shear forces having been applied for 30 minutes, 60 minutes, and 120 minutes respectively.

FIG. 17, panel B shows experimental data for IL-2 expression for fresh PBMCs by assessing IL-2 expression. Percentage IL-2 expression as a total of the cell population is shown on the y-axis and timepoints are shown on the x-axis. Columns 1, 2, and 3 show post shear force application IL-2 expression after 24 hours and 72 hours total with shear forces having been applied for 30 minutes, 60 minutes, and 120 minutes respectively. Columns 4, 5, and 6 show post shear force application IL-2 expression after 192, at harvest, hours total with shear forces having been applied for 30 minutes, 60 minutes, and 120 minutes respectively. A clear trend in increased IL-2 expression was observed at harvest.

FIG. 17, panel C shows experimental data for IL-2 expression for cryo preserved T cells by assessing IL-2 expression. Percentage IL-2 expression as a total of the cell population is shown on the y-axis and timepoints are shown on the x-axis. Columns 1, 2, and 3 show post shear force application IL-2 expression after 24 hours and 72 hours total with shear forces having been applied for 30 minutes, 60 minutes, and 120 minutes respectively. Columns 4, 5, and 6 show post shear force application IL-2 expression after 192, at harvest, hours total with shear forces having been applied for 30 minutes, 60 minutes, and 120 minutes respectively.

FIG. 17, panel D shows experimental data for IL-2 expression for cryo preserved PBMCs by assessing IL-2 expression. Percentage IL-2 expression as a total of the cell population is shown on the y-axis and timepoints are shown on the x-axis. Columns 1, 2, and 3 show post shear force application IL-2 expression after 24 hours and 72 hours total with shear forces having been applied for 30 minutes, 60 minutes, and 120 minutes respectively. Columns 4, 5, and 6 show post shear force application IL-2 expression after 192, at harvest, hours total with shear forces having been applied for 30 minutes, 60 minutes, and 120 minutes respectively. A clear trend in increased IL-2 expression was observed at harvest.

q. Experimental Conclusions

Starting material for both T cells and PBMCs in the fresh and cryo preserved conditions exhibited similar positive trends when exposed to peristaltic pumping at 400 mL/min. The extreme duration which exposed the cell culture to 120 minutes of continuous pumping had the most noticeable response, but lower shear force application rates also showed positive response.

Early T cell activation marker CD69 was the strongest in this condition while late activation marker CD25 was also expressed. Shear force application clearly drives the populations of cells to have the largest ratio of naïve and central memory T cells to effector and terminally differentiated T cells which is desirable for cell therapy products. CD4+/CD8+ cell ratios were improved showing memory T cells present in higher rates. Upregulated levels of PD-1 early in the T cell manufacturing process track with the increase T cell activation marker CD69. The PD-1 levels downregulate in line with the T cell late state activation marker CD25. The costimulatory markers CD27 and CD28 were not affected by peristaltic pumping at any duration. These markers were expressed similarly to the control condition further indicating no negative effect on the cells. Finally, at the end of the experiment, the IL-2 levels in solution were elevated for all experimental conditions indicating continuing cell proliferation which is also desirable in cell therapy products. Exposure to the extreme experimental conditions (e.g., 120 mins duration at 400 mL/min) show that T cell cultures placed in our custom mechanical force generation system benefit from the additional levels of fluidic shear stress without sacrificing cell viability.

Purified T cells and PBMCs from healthy donors demonstrated a response to high and extreme levels of shear stress exposure. Both starting material types (i.e. T cells and PBMCs) showed an increase in cell performance, activation, and expansion. Both fresh and cryo preserved starting material illustrated similar cell culture performance. The minor differences between the fresh and cryo preserved cell cultures may be due to the cellular membrane post freeze-thaw cycle. Additional differences can be attributed to varying donor starting material quality. It is difficult to establish a platform process with varying donor material so the universally improved conditions after applying high levels of shear force, as shown herein, will lead to improved cell therapy products overall.

The data generated opposes the consensus that cell cultures prefer lower amounts of fluidic shear stress and increasing it will eventually result in an impact to process performance. The methods described herein are applicable to a variety of different systems and may not be restricted to a specific device.

The established methods can be further applied throughout the process at different points or continuously throughout the process. Exposure at multiple points of the process may affect the cell culture positively. Cells under discontinuous exposure may benefit from the pulsing nature of the shear stress, similar to the environment that is found in a human body such as exposure during cell expansion.

T cell transduction can potentially be improved by allowing the cells to be exposed to shear stress. A peristaltic pump may physically deform the cell shape and help with the transduction process. The cells may be squeezed by the pump assembly and cause the membrane to deform, allowing movement of molecules across the membrane according to various embodiments.

Adding shear stress during T cell activation may help improve activation efficiency by triggering upregulation of TCR receptors and ensuring full activation occurs in the cells. The concept that T cells can sense forces that can trigger the activation pathway.

Finally, generating the positive response of CD4 and CD8 expression to produce an ideal ratio of CD4+/CD8+ cell types caused by application of fluidic shear force as described herein may lead to more positive clinical outcomes (e.g., better survival characteristics).

The data described in the Examples section above is summarized in the tables below.

TABLE 6A
PBMCs: Activation

| Cell | Shear | T Cell Activation | T Cell Activation |
|---|---|---|---|
| Condition | Level | CD69+ | CD25+ |
| Fresh | Normal | ↓1% | ↑5% |
| | High | ↑0% | ↓3% |
| | Extreme | ↓1% | ↑8% |
| Cyro | Normal | ↑4% | ↑0% |
| preserved | High | ↑4% | ↑0% |
| | Extreme | ↑5% | ↑0% |

TABLE 6B
T Cells: Activation

| Cell | Shear | T Cell Activation | T Cell Activation |
|---|---|---|---|
| Condition | Level | CD69+ | CD25+ |
| Fresh | Normal | ↑2% | ↑8% |
| | High | ↑2% | ↑12% |
| | Extreme | ↑4% | ↑14% |
| Cyro | Normal | ↑2% | ↑0% |
| preserved | High | ↑3% | ↓1% |
| | Extreme | ↑0% | ↓2% |

TABLE 7A
PBMCs: Phenotype

| | | T Cell | T Cell | T Cell | T Cell |
|---|---|---|---|---|---|
| Cell | Shear | Phenotype | Phenotype | Phenotype | Phenotype |
| Condition | Level | Naïve | Central | Effector | TEMRA |
| Fresh | Normal | ↑2.1% | ↑0.3% | ↓3% | ↑0.7% |
| | High | ↑2.3% | ↑3.2% | ↓6.1% | ↑0.7% |
| | Extreme | ↑1.2% | ↑3.8% | ↓5.4% | ↑0.6% |
| Cyro | Normal | ↑0% | ↑0.3% | ↓0.35% | ↓0.2% |
| preserved | High | ↑2.5% | ↓1.5% | ↓1.0% | ↑0% |
| | Extreme | ↑1.9% | ↓3.2% | ↑1.1% | ↑0.18% |

TABLE 7B
T Cells: Phenotype

| | | T Cell | T Cell | T Cell | T Cell |
|---|---|---|---|---|---|
| Cell | Shear | Phenotype | Phenotype | Phenotype | Phenotype |
| Condition | Level | Naïve | Central | Effector | TEMRA |
| Fresh | Normal | ↑0.3% | ↑4.3% | ↓4.8% | ↑0% |
| | High | ↑0% | ↑9.9% | ↓7.5% | ↑0% |
| | Extreme | ↑0.3% | ↑16.3% | ↓12.9% | ↑0% |
| Cyro | Normal | ↓0.5% | ↑2.9% | ↓2.3% | ↓0.16% |
| preserved | High | ↓0.7% | ↑1.5% | ↓0.7% | ↓0.05% |
| | Extreme | ↓3.8% | ↑4.2% | ↓0.2% | ↓0.15% |

TABLE 8A
PBMCs: CD4+/CD8+ Ratio

| Cell Condition | Shear Level | CD4+/CD8+ Ratios |
|---|---|---|
| Fresh | Normal | ↓0.08 |
| | High | ↓0.03 |
| | Extreme | ↓0.12 |
| Cyro preserved | Normal | ↑0 |
| | High | ↓0.04 |
| | Extreme | ↓0.02 |

TABLE 8B
T Cells: CD4+/CD8+ Ratio

| Cell Condition | Shear Level | CD4+/CD8+ Ratios |
|---|---|---|
| Fresh | Normal | ↑0.08 |
| | High | ↑0.07 |
| | Extreme | ↓0.02 |
| Cyro preserved | Normal | ↑0.02 |
| | High | ↑0.01 |
| | Extreme | ↑0.01 |

TABLE 9A
PBMCs: PD-1+, CD27+, CD28+, IL-2

| Cell | Shear | | | | |
|---|---|---|---|---|---|
| Condition | Level | PD-1+ | CD27+ | CD28+ | IL-2 |
| Fresh | Normal | ↓2.5% | ↓1% | ↓0.5% | ↑13.6% |
| | High | ↓7.6% | ↑1% | ↓1% | ↑19.8% |
| | Extreme | ↓11.6% | ↑2.5% | ↓1% | ↑22.3% |
| Cyro | Normal | ↑0.7% | ↓0.4% | ↓0.1% | ↑20.8% |
| preserved | High | ↑1.4% | ↑0% | ↓0.1% | ↑23.6% |
| | Extreme | ↑2.6% | ↑0% | ↑0% | ↑24.8% |

TABLE 9B
T Cells: PD-1+, CD27+, CD28+, IL-2

| Cell | Shear | | | | |
|---|---|---|---|---|---|
| Condition | Level | PD-1+ | CD27+ | CD28+ | IL-2 |
| Fresh | Normal | ↑4.5% | ↑1.8% | ↑2.2% | ↑23.3% |
| | High | ↑5.4% | ↑1.4% | ↑3.2% | ↑25.4% |
| | Extreme | ↑7.1% | ↑3.9% | ↑3.4% | ↑20.7% |
| Cyro | Normal | ↑0.6% | ↑1.2% | ↑0.4% | ↑19.7% |
| preserved | High | ↑1.5% | ↑0.5% | ↑0.3% | ↑31.5% |
| | Extreme | ↑2.6% | ↑0.3% | ↑0.3% | ↑21.8% |

TABLE 10
PBMCs: T Cells, B Cells, NK Cells

| Cell | Shear | | | |
|---|---|---|---|---|
| Condition | Level | T Cells | B Cells | NK Cells |
| Fresh | Normal | ↑4.2% | ↓2.4% | ↓1.0% |
| | High | ↑6.2% | ↓2.8% | ↓1.3% |
| | Extreme | ↑8.4% | ↓4.5% | ↓2.0% |
| Cyro | Normal | ↑2.1% | ↓0.3% | ↓0.3% |
| preserved | High | ↑6.3% | ↓0.5% | ↓0.5% |
| | Extreme | ↑10.2% | ↓1.8% | ↓1.1% |

### EQUIVALENCE

Those skilled in the art will recognize or be able to ascertain using no more than routine experimentation, many equivalents to the specified embodiments of the technologies described herein. It is to be understood that the technologies encompass all variants, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the technologies can be explicitly excluded from the claims, regardless of whether the specific exclusion is recited in the specification. The scope of the present disclosure is not limited to the description herein, but rather is as set forth in the claims below.

1. A method of applying a shear force to a first cell population to cause a subsequent phenotypic response, the method comprising:
delivering a fluid comprising a first cell population to a mechanical force generating system;
applying a shear force to the first cell population using the mechanical force generating system to create a second cell population, wherein the step of applying the shear force causes a subsequent phenotypic response;
activating the second cell population to create an activated cell population;
transducing the activated cell population to create a final cell population; and
harvesting the final cell population to generate a cell therapy product, wherein the subsequent phenotypic response comprises an increase of central memory or naïve cells in the final cell population.

2. The method of claim 1, wherein the increase of central memory cells is 0.3% to 16.3% and the increase of naïve cells is 0.3% to 2.5%.

3. The method of claim 1, wherein the subsequent phenotypic response comprises a decrease of effector cells in the final cell population.

4. The method of claim 3, wherein the decrease of effector cells is 0.2% to 12.9%.

5. The method of claim 1, wherein the subsequent phenotypic response comprises an increase of T cells and a decrease of both B cells and NK cells in the final cell population.

6. The method of claim 5, wherein the increase of T cells is 2.1% to 10.2%, the decrease of B cells is 0.3% to 4.5% and the decrease of NK cells is 0.3% to 2.0%.

7. The method of claim 1, wherein the subsequent phenotypic response comprises an increase of CD69 or CD25 expression in the activated cell population.

8. The method of claim 7, wherein the increase of CD69 is 2% to 4% and the increase of CD25 is 5% to 14%.

9. The method of claim 1, wherein the subsequent phenotypic response comprises an increase of PD-1 expression.

10. The method of claim 9, wherein the increase of PD-1 expression is 0.6% to 7.1%.

11. The method of claim 1, wherein the subsequent phenotypic response comprises an increase of CD27 or CD28 expression.

12. The method of claim 11, wherein the increase of CD27 expression is 0.3% to 3.9% and the increase of CD28 expression is 0.3% to 3.4%.

13. The method of claim 1, wherein the step of transducing comprises using a lentiviral vector or a retroviral vector.

14. The method of claim 1, wherein the first cell population comprises T cells.

15. The method of claim 1, wherein the first cell population comprises peripheral blood mononuclear cells (PBMCs).

16. The method of claim 1, wherein the step of applying the shear force occurs for 30 minutes to 120 minutes.

17. The method of claim 1, wherein the step of applying the shear force occurs for 60 minutes to 120 minutes.

18. The method of claim 1, wherein the step of applying the shear force comprises applying at a shear rate of 569.78 s⁻¹ to 10,533.53 s⁻¹.

19. The method of claim 1, wherein the step of applying the shear force comprising applying at a shear rate of 614.75 s⁻¹.

20. The method of claim 1, wherein the step of applying the shear force comprises applying a total shear exposure of 1,106,557 s to 4,426,230 s.

21. The method of claim 1, wherein the mechanical force generating system comprises:
a housing for storing the cell population;
a fluid channel having a first end and a second end, wherein the first and second ends are fluidically connected to the housing; and
a pump positioned along the fluid channel or in the housing.

22. The method of claim 21, wherein the inner diameter of the fluid channel is 4.8 mm to 25.4 mm.

23. The method of claim 21, wherein the length of the fluid channel is 45 cm.

24. The method of claim 21, wherein the pump operates to produce a fluid flow rate of 400 mL/min to 55,000 mL/min.

25. The method of claim 21, wherein the pump comprises a peristaltic pump.

26. An improved cell therapy product made by:
delivering a fluid comprising a first cell population to a mechanical force generating system;
applying a shear force to the first cell population using the mechanical force generating system to create a second cell population, wherein the step of applying of the shear force causes a subsequent phenotypic response;
activating the second cell population to create an activated cell population;
transducing the activated cell population to create a final cell population; and
harvesting a final cell population to generate a cell therapy product, wherein the subsequent phenotypic response comprises an increase of central memory or naïve cells in the final cell population.

27. An improved cell therapy product, wherein the improved cell therapy product comprises a population of shear force treated cells having an increase of CD69 expression, CD25 expression, PD-1 expression, CD27 expression, CD28 expression, IL-2 expression, CD4 expression, CD8 expression, naïve T-cells, central memory T cells, central memory T cells, or a combination thereof.

28. A method of applying a shear force to a first cell population to cause a subsequent phenotypic response, the method comprising:
delivering a fluid comprising a first cell population to a mechanical force generating system;
step for applying a shear force to the first cell population using the mechanical force generating system to create a second cell population, wherein the step for applying the shear force causes a subsequent phenotypic response;
activating the second cell population to create an activated cell population;
transducing the activated cell population to create a final cell population; and
harvesting a final cell population to generate a cell therapy product, wherein the subsequent phenotypic response comprises an increase of central memory or naïve cells in the final cell population.
